(19) United States
(12) Patent Application Publication
(10) Pub. No.: US 2002/0107433 A1
(43) Pub. Date: Aug. 8, 2002

(54) System and method of personal fitness training using interactive television

(76) Inventor: James R. Mault, Evergreen, CO (US)

Correspondence Address: Gifford, Krass, Groh, Sprinkle, Anderson & Citkowski, P.C., Suite 400, 280 N. Old Woodward Avenue, Birmingham, MI, 48009, US

(21) Appl. No.: 10/078,044

(22) Filed: Feb. 19, 2002

Related U.S. Application Data
(63) Continuation-in-part of application No. 09/685,625, filed on Oct. 10, 2000.
(60) Provisional application No. 60/158,553, filed on Oct. 8, 1999.
(60) Provisional application No. 60/167,276, filed on Nov. 24, 1999.
(60) Provisional application No. 60/177,016, filed on Jan. 19, 2000.
(60) Provisional application No. 60/178,979, filed on Jan. 28, 2000.
(60) Provisional application No. 60/194,126, filed on Apr. 3, 2000.
(60) Provisional application No. 60/200,428, filed on Apr. 28, 2000.
(60) Provisional application No. 60/207,051, filed on May 25, 2000.
(60) Provisional application No. 60/207,089, filed on May 25, 2000.
(60) Provisional application No. 60/209,921, filed on Jun. 7, 2000.
(60) Provisional application No. 60/219,069, filed on Jul. 18, 2000.
(60) Provisional application No. 60/219,512, filed on Jul. 20, 2000.
(60) Provisional application No. 60/228,680, filed on Aug. 29, 2000.

(51) Int. Cl. A61B 5/00

(52) U.S. Cl. 600/300; 128/920; 482/9; 348/14.05; 725/73

(57) ABSTRACT

An improved system and method of health management using a personal fitness trainer and interactive television is provided. The system includes an entertainment device, a control means, a remote control device, and an interactive television network in communication with the control means via a communication network. The system also includes a computer system in communication with the interactive television network, and the interactive television network transmits a signal representing a personal fitness training program for the user that is processed and displayed on the display screen of the entertainment device so that the user can interactively participate in the personal fitness training program. The method includes the steps of the user selecting the personal fitness training program using the remote control device and selecting a transactional selection from a predetermined list for the personal fitness training program displayed on the display screen for the entertainment device. The method also includes the steps of the interactive television network processing the selection and displaying the appropriate information on the display screen of the entertainment device. The method further includes the step of the user interactively participating in the program via the interactive television network.

### RELATED APPLICATIONS

This application is a continuation-in-part of patent application Ser. No. 09/685,625 filed Oct. 10, 2000, which claims the benefit of provisional patent applications Ser. No. 60/158,553 filed Oct. 8, 1999; Ser. No. 60/167,276 filed Nov. 24, 1999; Ser. No. 60/177,016 filed Jan. 19, 2000; Ser. No. 60/177,016 filed Jan. 19, 2000; Ser. No. 60/178,979 filed Jan. 28, 2000; Ser. No. 60/194,126 filed Apr. 3, 2000; Ser. No. 60/200,428 filed Apr. 28, 2000; Ser. No. 60/207,051 filed May 25, 2000; Ser. No. 60/207,089 filed May 25, 2000; Ser. No. 60/209,921 filed Jun. 7, 2000; Ser. No. 60/219,069 filed Jul. 18, 2000; Ser. No. 60/219,512 filed Jul. 20, 2000; Ser. No. 60/228,680 filed Aug. 29, 2000, which are hereby incorporated by reference.

### FIELD OF THE INVENTION

This invention relates to health management, and in particular, to a system and method of personal fitness training using interactive television.

### BACKGROUND OF THE INVENTION

Health management involves balancing both the emotional and physical needs of an individual, and these needs are frequently interrelated. Various health management tools are known to achieve health management goals, including weight control and activity level. A personalized health management program that balances the individual's needs and goals enhances the success of such a program. To achieve a personal fitness goal, a personal fitness trainer is used to assist in developing and maintaining a personal exercise program. However, it is expensive to utilize a personal fitness trainer on an ongoing basis.

Individuals also engage in conventional weight loss schemes, usually based on a restricted calorie diet, to achieve a personal weight loss goal. For example, a calorie management system allows a person to compare their caloric expenditure, comprising resting metabolic rate (RMR) and activity-related caloric expenditure, to their caloric intake in the form of food and beverages. Caloric expenditure has two components, a larger contribution related to resting metabolic processes, and a smaller contribution related to the energy expended in physical activity. Total energy expenditure (TEE) is the sum of resting energy expenditure (REE, a product of resting metabolic rate and the time period of interest) and activity-related energy expenditure (AEE), i.e.:

TEE=REE+AEE

Caloric balance is defined in terms of the difference between TEE and the caloric intake of the person.

In some prior art systems of weight management, also referred to as weight control, a person's RMR has been estimated using the Harris-Benedict equation, which relates RMR to body height, weight, age, and gender. This equation is well known to those skilled in the diet and nutrition arts (e.g. Williams, certificate of correction to U.S. Pat. No. 5,704,350, and Krause and Mahon, “Food Nutrition and Diet Therapy”). Alternatively, charts and tables, usually based on the Harris-Benedict equation, may be used. Additional demographic factors and body fat percentage may be included to improve the estimate of RMR.

It is important to realize that the values of RMR obtained using equations, tables, charts and the like, only provide an estimated RMR value for an average person. A person with a given height, weight, or other physical parameter (such as may be entered into the Harris-Benedict equation or modified equation) may have an actual RMR that is significantly different from the estimate. Actual RMR values for individuals within a group of apparently similar persons will fall on a distribution around the estimated RMR value for an average person. This distribution leads to errors in the caloric needs calculated for a person in a weight control program.

Additionally, there is an even more serious inadequacy in conventional weight control programs. The RMR of a person changes unpredictably as a weight control program progresses. A person may respond to the perceived starvation conditions through a significant drop in RMR. As a consequence, such a person may even gain weight on a reduced calorie diet if their caloric intake required to maintain a given weight falls below the reduced value prescribed by the diet. This is an unsatisfactory outcome to a weight control program. Other people may suffer no fall in metabolic rate during the restricted calorie diet. If the weight control program contains an exercise component, the resting metabolic rate of a person may even increase during the program. The Harris-Benedict equation predicts that resting metabolism will fall as body weight is lost, but is not intended to predict the actual response of a person's resting metabolic rate to a weight control program. Hence, estimating the resting metabolic rate of a person using an equation may lead to large errors in calculating the caloric needs and activity levels required for an effective weight control program. Hence, an improved weight control method which accurately compensates for changes in metabolic rate over time will be of great value.

RMR can be determined using an indirect calorimeter. Conventional devices are large, expensive, and difficult to use so that expert assistance is essential. A person will need to report to a specific location, such as a hospital, for use of a conventional indirect calorimeter. There is considerable difficulty and expense associated with conventional indirect calorimeter use, so that conventional weight loss programs do not monitor the RMR of the person in the program, but rather rely on an estimate such as provided by the Harris-Benedict equation.

A very large number of weight loss approaches have been proposed, all of which suffer from the above discussed shortcomings. For example, in U.S. Pat. No. 5,704,350, Williams describes a nutritional microcomputer and method of use in a weight control program. A handheld device is described which enables a diet log to be recorded, activity levels to be recorded, and diet goals to be set. The Harris-Benedict equation is used to calculate the user's daily caloric expenditure. Hence this device and method fails to take into account the change in RMR at the onset of a diet.

In U.S. Pat. No. 5,673,691, Abrams et al. describe an apparatus to control weight, in which caloric intake levels are adjusted on the basis of changes in the user's body weight. The actual metabolic rate of the user is not determined in the described method of using this device.

In U.S. Pat. No. 4,951,197, Mellinger describes a diet method in which caloric expenditure is calculated from the weight of the person. Individual variations in RMR, and RMR changes during a diet, are not taken into account.

In U.S. Pat. No. 5,890,128, Diaz et al. describe a handheld calorie computer for use in a weight control program. For weight loss, caloric intake is decreased gradually so as to hopefully avoid abrupt changes in the user's metabolic rate. However, this is not as effective as actually measuring the user's metabolic rate and compensating for changes, as described in embodiments of the present invention.

In U.S. Pat. No. 5,705,735, Acorn describes monitoring the oxygen consumption and carbon dioxide production of a patient on a ventilator, and using the data to assess nutritional requirements. This apparatus not intended to provide information to the patient, but rather to a health professional in attendance, and is not convenient for use in a weight control program.

In U.S. Pat. No. 5,989,188, Birkhoelzer et al. describe the use of indirect calorimetrics in determining the energy balance of a living subject. However, Birkhoelzer et al. do not envision the problematical effects of metabolic change caused by a weight control program on predicting the outcome of the weight control program. They do not describe a weight control program in which the RMR of the subject is monitored through the course of the program, and do not describe how changes in RMR may be used to modify the recommended caloric intake, activity levels, and/or target goals of a weight control program.

In commonly assigned U.S. Ser. No. 09/630,398, and incorporated herein by reference, many of the shortfalls of the above-described techniques for caloric measurement are improved upon. U.S. Ser. No. 09/630,398 discloses a low-cost, handheld, portable indirect calorimeter, referred to as a Gas Exchange Monitor (GEM). This device allows accurate measurement of resting metabolic rate (RMR). Advantageously, the periodic use of the GEM to measure the RMR of a person in a weight control program, monitors changes in RMR, which cannot be accounted for by weight loss using the Harris-Benedict equation.

For example, the GEM can be used to measure the resting metabolic rate (RMR) of a person at intervals, and modify the RMR component of caloric balance on a dynamic basis to compensate for changes in metabolism which occur during weight control, particularly weight loss.

In the weight control program described in copending U.S. Ser. No. 09/685,625, and incorporated herein by reference, an indirect calorimeter is used to monitor the RMR of a person at intervals. The RMR values are used to modify the caloric intake and/or activity levels recommended in the weight control program.

The GEM allows direct measurement, not an estimate, of a person's RMR at intervals as a person's metabolism changes as a result of a weight control program. RMR changes may be accurately tracked over the course of a weight loss program. RMR may be measured at more frequent intervals (for example, once every 1-5 days) at the start of a weight control program, when metabolism changes may be more rapid. The measurement intervals may be lengthened (for example to every 1-4 weeks) if the person's RMR settles down to an approximately constant value in the course of a weight control program.

In a conventional weight loss program, a person will often become discouraged due to small or nonexistent actual weight losses. This is often due to a failure to take RMR changes into account. If the user knows their RMR at a given time, they can adjust their lifestyle accordingly, such as by reducing caloric intake, increasing activity levels, or modifying weight loss expectations, take into account the changing value of RMR.

In one embodiment of the present invention, a person is provided with a portable computing device, such as a personal digital assistant, with software which enables the device to function as a caloric intake calculator, a caloric expenditure calculator, and a caloric balance calculator. A body weight target may be set, and the initial RMR value is used to suggest a caloric intake level and activity level by which the target weight may be achieved in a reasonable time. Soon after the start of the weight control program, the person may be prompted or otherwise reminded to re-determine their RMR level. RMR may change significantly at the beginning of a weight control program. Any significant changes in RMR may be used to recalculate a reasonable balance of caloric intake, caloric expenditure, and time needed to reach a certain body weight goal. The RMR of the person is measured at intervals through the duration of the weight control program, so as to revise the parameters of the program in a manner consistent with a successful outcome.

While a caloric balance log works well as one aspect of a health management program, personal fitness training is another important aspect. At the same time, the explosive growth of the internet, and in particular, the Internet, provides greater opportunities for interaction between individuals that are remotely located. In addition, the emerging field of interactive television provides further opportunities for individuals to interact. For example, in co-pending U.S. Ser. No. 09/995,952, and incorporated herein by reference, the use of interactive television as part of a health management program is described. While this system works well, it is enhanced by a personal fitness trainer. Thus, there is a need in the art for a system and method of personal fitness training using interactive television as part of an individual's health management program.

### SUMMARY OF THE INVENTION

Accordingly, the present invention is an improved system and method of health management using a personal fitness trainer and interactive television. The system includes an entertainment device, a control means in communication with the entertainment device, a remote control device having a user input mechanism, and an interactive television network in communication with the control means via a communications network. The system also includes a computer system in communication with the interactive television network, and the interactive television network transmits a signal representing a personal fitness training program developed by the personal fitness trainer for the user that is processed and displayed on the display screen of the entertainment device so that the user can interactively participate in the personal fitness training program. The method includes the steps of the user selecting the personal fitness training program using the remote control device and selecting a transactional selection from a predetermined list for the personal fitness training program displayed on the display screen for the entertainment device. The method also includes the steps of the interactive television network processing the selection and displaying the appropriate information on the display screen of the entertainment device. The method further includes the step of the user interactively participating in the program via the interactive television network using the remote control device, control means and entertainment device.

One advantage of the present invention of a system and method of personal fitness training using interactive television is provided that allows the user to actively manage their health on an ongoing basis. Another advantage of the present invention is that the resting metabolic rate (RMR) of a person is measured at intervals, and the RMR component of caloric balance is modified on a dynamic basis to compensate for changes in metabolism which occur during weight management, and in particularly weight loss. Still another advantage of the present invention is that an indirect calorimeter is used to monitor the RMR of a person at intervals, and the RMR values are used to modify the caloric intake and/or activity levels recommended as part of the personal fitness training program. A further advantage of the present invention is that the user uses interactive television to communicate with a personal fitness trainer as part of a personalized personal fitness training program.

Other features and advantages of the present invention will be readily appreciated, as the same becomes better understood after reading the subsequent description taken in conjunction with the accompanying drawings.

### BRIEF DESCRIPTION OF THE DRAWINGS

FIG. 1 is a perspective view of a user breathing through an indirect calorimeter according to the present invention;

FIG. 2 is a perspective view of an indirect calorimeter;

FIG. 3 is a cross-sectional view of an indirect calorimeter;

FIG. 4 is a schematic of a system embodiment of the present invention;

FIG. 5 illustrates an indirect calorimeter in communication with a portable computing device according to the present invention;

FIG. 6 is a flowchart of software which may run on a computing device, according to embodiments of the present invention;

FIGS. 7-12 illustrate example screens provided by software running on a computing device;

FIG. 13 is a schematic of a system embodiment of the present invention, including an activity sensor;

FIGS. 14A and 14B show a user carrying an activity sensor according to the present invention;

FIG. 15 is a schematic of another system embodiment of the present invention;

FIG. 16 is a schematic of another system embodiment, by which improved feedback may be provided to the user;

FIG. 17 shows a wrist-mounted computing device according to the present invention;

FIG. 18 illustrates a user breathing through an indirect calorimeter in communication with a desktop computer;

FIG. 19 is a schematic diagram of a system of integrated calorie management using interactive television, according to the present invention;

FIG. 20 is a flowchart of a method of integrated calorie management using the system of FIG. 19, according to the present invention;

FIGS. 21-29 illustrate example screens for the method of FIG. 20, according to the present invention;

FIG. 30 is a schematic diagram of a system of personal fitness training using interactive television, according to the present invention;

FIG. 31 is a flowchart of a method of personal fitness training using the system of FIG. 30, according to the present invention; and

FIGS. 32-38 illustrate example screens for the method of FIG. 31, according to the present invention.

### DETAILED DESCRIPTION OF THE INVENTION

Health management involves many interrelated factors, including weight control and activity level. A person's caloric balance is the difference between their caloric intake (from food, beverages, and other consumables) and their caloric expenditure. Hence, an effective calorie management system has two components. It must monitor caloric intake, and must also monitor caloric expenditure in terms of resting metabolism and physical activity levels.

Various types of diet logging software and activity sensors are known in the art. However, conventional weight control programs do not compensate for changes in resting metabolism during the course of a weight control program. In the improved weight control program described here, an indirect calorimeter is used to measure the RMR of a person at intervals. The values of RMR are then used in a calorie management system.

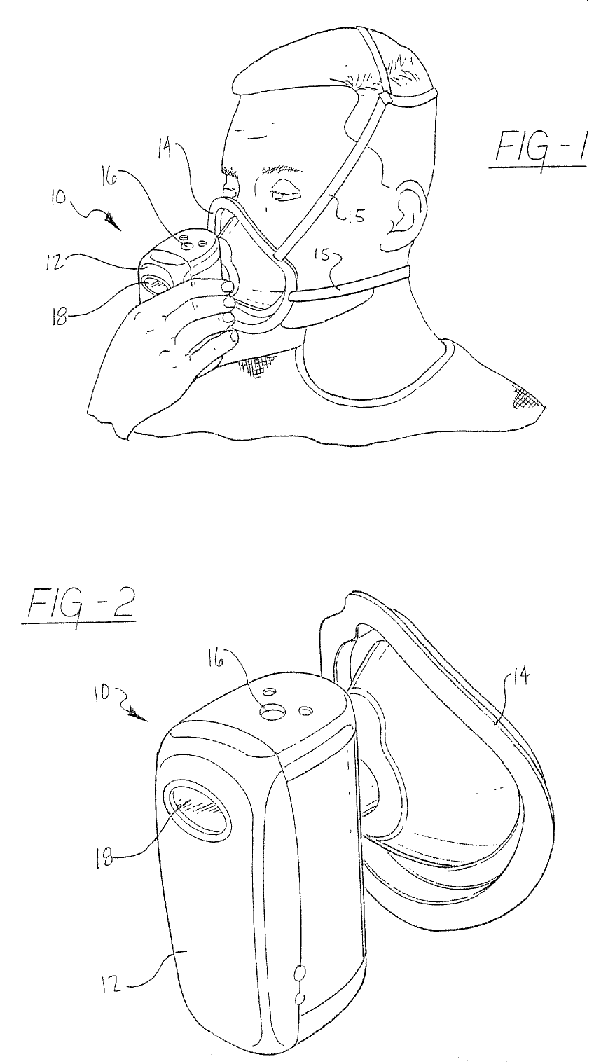

The gas exchange monitor (GEM) disclosed in U.S. Ser. No. 09/630,398, is preferably used in the health management program described here. Referring to FIGS. 1 and 2, the calorimeter according to U.S. patent application Ser. No. 09/630,398 is generally shown at 10. The calorimeter 10 includes a body 12 and a respiratory connector, such as mask 14, extending from the body 12. In use, the body 12 is grasped in the hand of a user and the mask 14 is brought into contact with the user's face so as to surround their mouth and nose, as best shown in FIG. 1. An optional pair of straps 15 is also shown in FIG. 1. With the mask 14 in contact with their face, the user breathes normally through the calorimeter 10 for a period of time. The calorimeter 10 measures a variety of factors and calculates one or more respiratory parameters, such as oxygen consumption and metabolic rate. A power button 16 is located on the top side of the calorimeter 10 and allows the user to control the calorimeter's functions. A display screen is disposed behind lens 18 on the side of the calorimeter body 12 opposite the mask 14. Test results are displayed on the screen following a test.

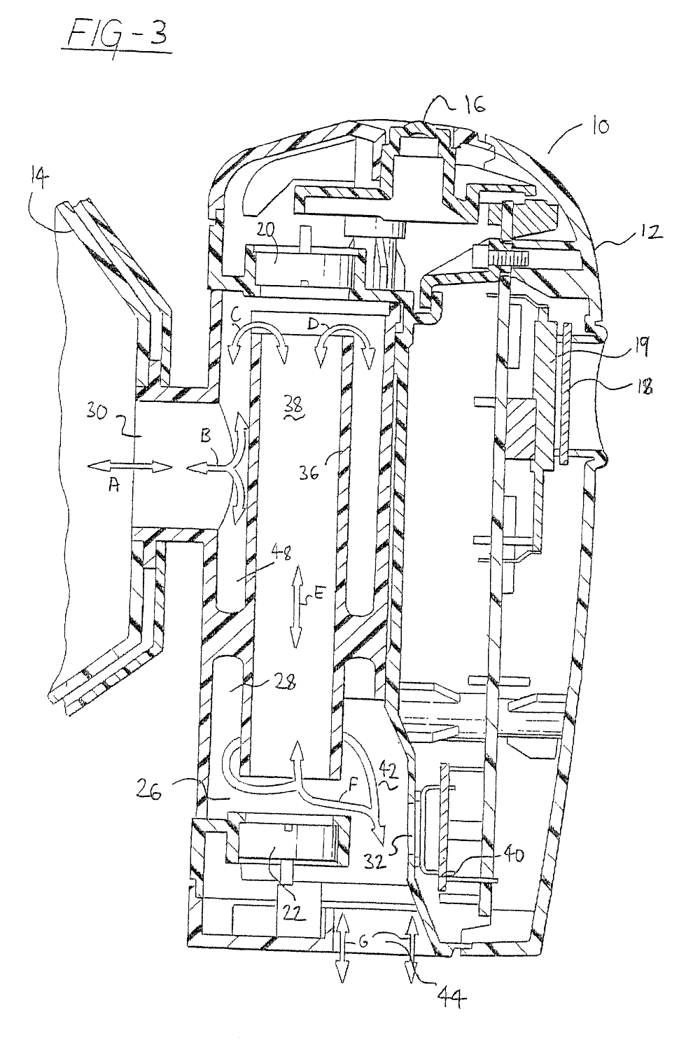

FIG. 3 shows a vertical cross section of the calorimeter 10. The flow path for respiration gases through the calorimeter 10 is illustrated by arrows A-G. In use, when a user exhales, their exhalation passes through the mask 14, through the calorimeter 10, and out to ambient air. Upon inhalation, ambient air is drawn into and through the calorimeter and through the respiratory connector to the user.

Exhaled air passes through inlet conduit 30, and enters connected concentric chamber 48. Excess moisture in a user's exhalations tends to drop out of the exhalation flow and fall to the lower end of the concentric chamber 48. Concentric chamber 48 serves to introduce the respiration gases to the flow tube 36 from all radial directions as evenly as possible. Exhaled air flows downwardly through a flow path 38 formed by the inside surface of the flow tube 36. Exhaled air enters outlet flow passage 42, via concentric chamber 28, and passes through the grill 44 to ambient air.

Flow rates through the flow path 38 are determined using a pair of ultrasonic transducers 20 and 22. An oxygen sensor 40, in contact with respiratory gas flow through opening 32, is used to measure the partial pressure of oxygen in the gas flow. Integration of oxygen concentration and flow rate allows inhaled oxygen volume and exhaled oxygen volume to be determined. The metabolic rate of the user is determined from the net oxygen consumption; the difference between inhaled and exhaled oxygen volumes. Metabolic rate is determined using either a measured or assumed respiratory quotient (the ratio of oxygen consumption to carbon dioxide production). For a user at rest, the RMR (resting metabolic rate) is determined. The RMR value is shown on display 19.

Preferably, the indirect calorimeter used in embodiments of the present invention comprises a respiratory connector such as a mask or mouthpiece, so as to pass respiration gases as the subject breathes; a flow pathway between the respiratory connector and a source and sink of respiratory gases (such as the atmosphere) which receives and passes the respiration gases; a flow meter configured to generate electrical signals as a function of the instantaneous flow of respiration gases passing through the flow pathway, such as an ultrasonic flow meter; and a component gas concentration sensor, such as a fluorescent oxygen sensor, which generates electrical signals as a function of the instantaneous fraction of gases such as oxygen and/or carbon dioxide in the respiration gases they pass through the flow pathway, such as the indirect calorimeter described above. However, other indirect calorimeters may be used in embodiments of the present invention, for example such as described in U.S. Pat. Nos. 4,917,108; 5,038,792; 5,178,155; 5,179,958; and 5,836,300, all to Mault, which are incorporated herein in their entirety by reference.

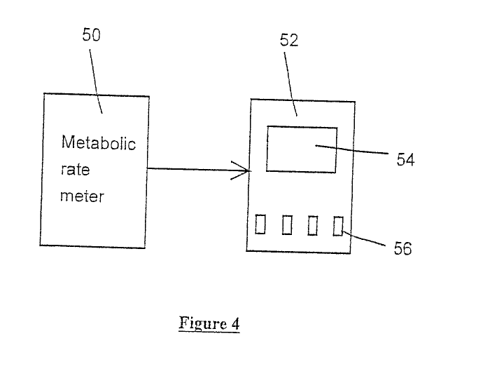

FIG. 4 shows a system embodiment of the present invention. The person using the system shown in FIG. 4, for example as part of a weight control program, is referred to as the user. A device for the measurement of metabolic rate (a metabolic rate meter) 50 provides metabolic rate data relating to the user at intervals to computing device 52. Preferably, an indirect calorimeter (such as that described in U.S. patent application Ser. No. 09/630,398) provides RMR measurements of the user to computing device 52. Device 52 has a display 54 and buttons 56. Buttons 56 may be used for data input (for example navigation through menus, character entry, and the like), changing the operating mode of the device (for example between computer and other functionality such as wireless phone), initiating a voice record, initiating an image capture, or other processes. Data entry may also be achieved using a stylus, touch-screen, roller-jog mechanism, touch-sensitive pad, monitoring eye movements, voice recognition software, barcode scanning, optical character recognition, or other convenient data entry mechanism. Preferably, computing device 52 is a personal digital assistant (PDA), but may be any electronic device such as a portable computer; electronic organizer; e-book; wireless phone; pager; wristwatch with added functionality; electronic system such as a system having separate display, entry, and computing modules; any portable/wearable device; a pedometer with added computing functionality; or a desktop computer system.

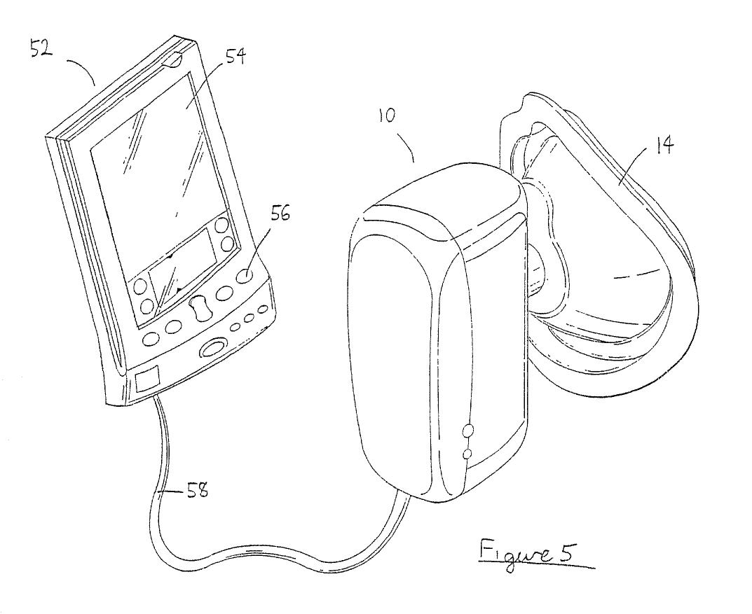

FIG. 5 shows an indirect calorimeter 10, with mask 14, in communication with a computing device 52 (with display 54 and data entry buttons 56) using a cable 58. FIG. 5 shows one embodiment of the invention, in which a personal digital assistant (PDA) is used as the computing device. Alternatively, a memory module (such as a memory card, memory stick, flash media, or the like) may be used to transfer data from the indirect calorimeter 10 to computing device 52. A wireless communication method, such as an IR beam or Bluetooth wireless protocol, may also be used.

Health management software running on computing device 52 receives the metabolic rate data at intervals, caloric intake data relating to diet, and physical activity level data. The software provides goals and feedback to the user in relation to weight goals, which are modified by changing values in the metabolic rate of the user. Diet logging software and activity sensors are known in the art. However, conventional weight control methods do not compensate for changes in the metabolic rate of the user over time. The overall capabilities of the software may be summarized in the following list: setting up a user identity by entering name and other information; setting of targets and goals based on information gathered from the user during an initial setup process (weight goals, nutrient targets, health goals, and activity plans); entry of food consumption through a food log with a search capability; entry of activity information combined with a search tool (alternatively using data from an activity sensor); feedback to the user regarding the caloric balance and time dependent logging of body measurements such as resting metabolism, body weight, and body fat percentage; reporting on body measurement trends using graphical display capabilities of the computing device or other device such as an interactive television; and reporting on the nutritional balance of food intake.

Software running on electronic device 52 preferably enables the device to function as a caloric intake calculator (allowing the user to enter data such as food item identity, indicative of food items (including beverages) consumed); a caloric expenditure calculator (allowing caloric expenditure to be determined from data related to physical activity of the user); and allows the device to receive RMR data related to the person at intervals. Activity data may be input by the user, either a numerical value associated with an activity, or by entering the type of activity such as through a menu based system. The user may set health related goals, such as body fat percentage, RMR, or other physiological parameters such as resting heart rate.

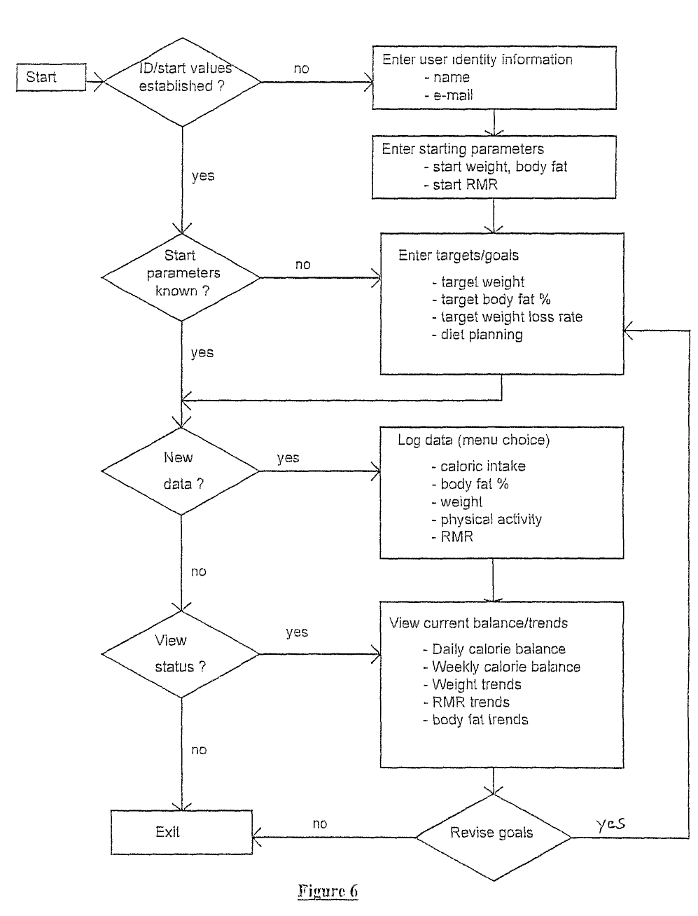

FIG. 6 shows a schematic illustration of one embodiment of health management software also referred to as a balance log, which may run on the computing device 52. At the start of a weight control program assisted by the health management software, the user may enter a setup procedure in which an identity is established, initial conditions entered, and targets and goals are set. Personal details such as name, e-mail address, birth date or age, gender, and other information such as frame size, and body fat percentage, may be entered into the software. The user then enters weight loss (or weight gain, or weight maintenance goals). The intended rate of weight loss may be entered, or may default to (for example) one pound per week. The user determines their resting metabolic rate using an indirect calorimeter, preferably the Gas Exchange Monitor disclosed in U.S. Ser. No. 09/630,398. The user also enters their lifestyle, sleep time, and typical exercise level into the device. The software then prepares an estimate or preview of the caloric balance for the person, indicating the caloric expenditure through RMR, caloric expenditure through activity, and caloric intake allowable by consumption. The user may adjust their intended activity level during the course of the weight loss program. The user then selects a customized diet using software on the computing device which allows a preferred distribution of carbohydrate, fat, and protein to be consumed.

During the course of a weight control program, the user enters foods consumed into the software. The food database accessed by the software preferably includes broad categories of food such as meat, vegetables, beverages, etc. and detailed subcategories related to the specific food items and their weight or volumes. Preferably, the food database resides on memory within the computing device 52. The food database may be created or enhanced using data received over a communications network, data received using a cable or wireless link to another device, or by transfer of memory modules. The computing device 52 may be supplied to customers by a weight control business with a food database pre-installed. The software may provide advice on future diet planning, for example suggesting lists of alternative foods which assist the user in achieving a weight loss goal. Activity levels may also be entered through a menu system. The computing device 52 preferably displays information related to the user's caloric and nutritional intakes, and displays trends, caloric balance, and other information relation to goals of the weight loss program.

The computing device then allows the user to view a breakdown of their daily caloric intake and intake of various food groups, vitamins, and minerals, which may be derived from current medical knowledge of healthy diets. After the setup is complete, the user enters diet information through a menu system. The user may select between various food groups to enter the identity of foods consumed. Activity level data is also supplied to the computing device either through user entry or information received from activity sensors. The consumption information and activity levels may then be transferred to a remote computer system. The device may be used as a progress calculator, by which the progress made towards target goals can be compared with initial projections.

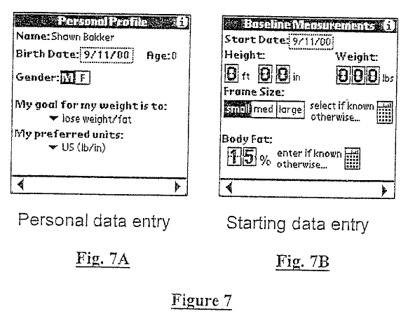

By way of illustration, FIGS. 7-12 show a number of example screens which may be shown on the display 54 of computing device 52, provided by health management software running on device 52. FIG. 7 shows two screens in which personal data and starting body parameters may be entered. FIG. 7A shows a personal data entry screen, FIG. 7B shows a starting data entry screen.

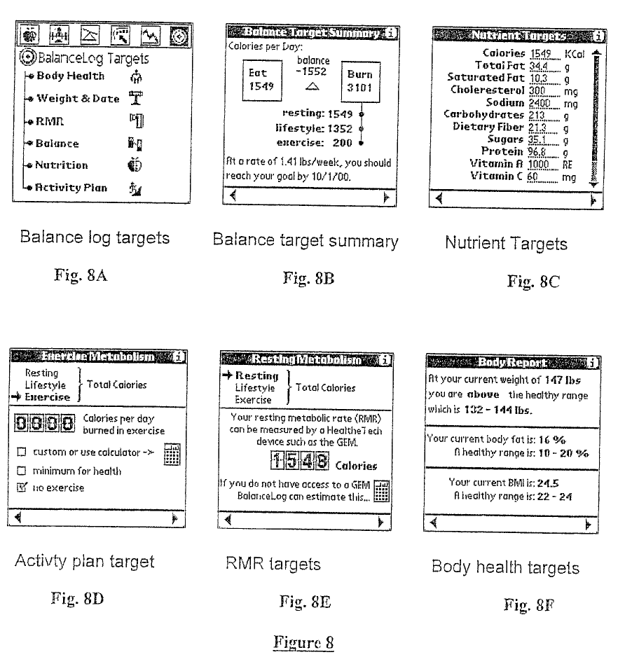

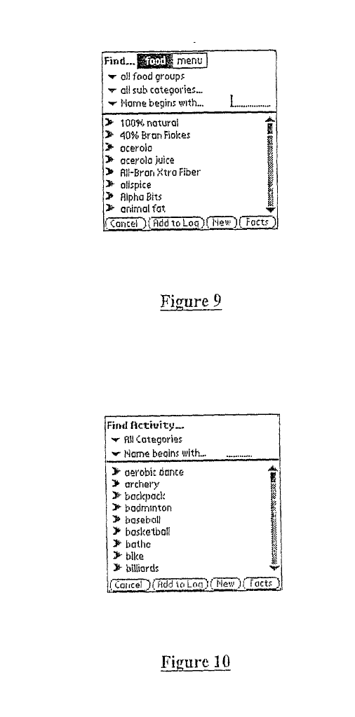

FIGS. 8A-8F show six screens by which weight control, activity, RMR, and nutritional targets may be displayed to the user, and/or adjusted, at the onset of the weight control program. FIG. 8A shows a menu screen from which other displays may be chosen, FIG. 8B shows a daily caloric balance target, FIG. 8C shows a daily nutrition target, FIG. 8D shows a screen in which activity levels may be entered, FIG. 8E shows the daily calories burned by the user's RMR, and FIG. 8F shows body health targets.

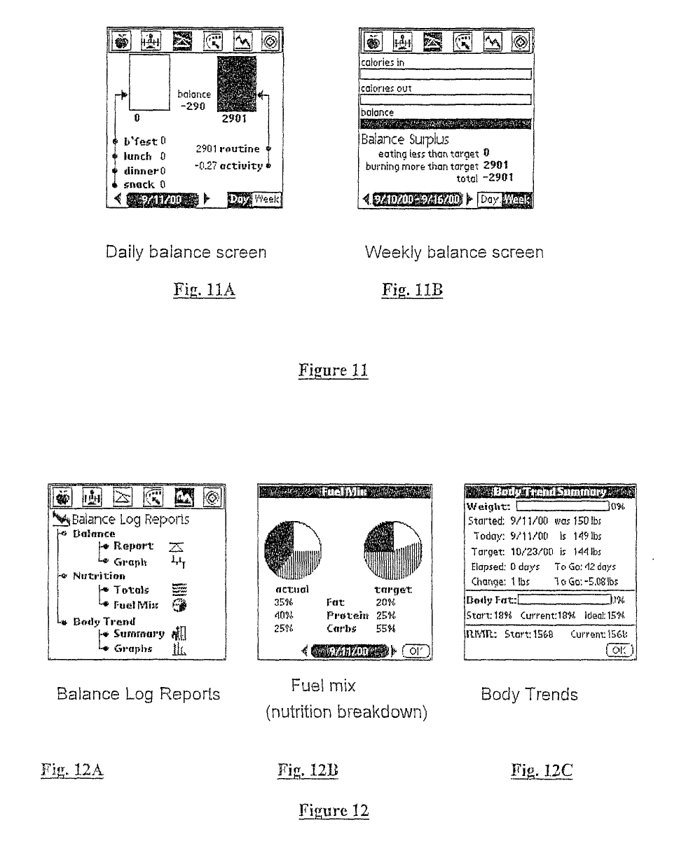

FIG. 9 shows a food database screen, allowing the user to enter diet choices. Names may be entered directly (by entering the first few letters), or through a menu system. Food products may be sorted by category or brand name. Also, the computing device 52 may be equipped with a barcode scanner by which product codes may be scanned off food packages, and the information obtained entered into a diet log database.

FIG. 10 shows an exercise database screen, allowing the user to enter or estimate activity levels. Activities may be entered directly by entering the first few letters of the name, or by selecting from menu options. An exercise database is preferably stored within the memory of the computing device 52, which relates activities to caloric expenditure.

FIG. 11 shows daily (FIG. 11A) and weekly (FIG. 11B) balance screens, allowing the user to view their caloric balance on a daily or weekly basis.

FIG. 12 shows three example screens, by which status aspects of the weight loss program may be observed. FIG. 12A shows a balance log report screen, allowing the options of tabular and graphical display of trends. FIG. 12B illustrates a screen showing nutrition breakdown, and FIG. 12C illustrates a screen showing body trends.

Weight control related data may be compared with previous day's, week's, or month's data, and trends determined. The day's caloric intake may be compared with that allowed for the successful achievement of the target weight. Changes that may relate to medical problems may be diagnosed, and the user's physician notified. The status of a person's caloric balance may be indicated by icons or screen displays based on various themes. For example, using a weather scheme, a cloud may be used to indicate caloric intake greater than caloric expenditure (which we may call a negative caloric balance), and a sunny sky used to indicate a caloric intake less than caloric expenditure (which we may call a positive caloric balance). Using a banking theme, an array of banknotes or other wealth representation may be used to show indicate caloric intake less than caloric expenditure, whereas pennies, a bill, or other representation of poverty may be used to indicate a caloric intake greater than caloric expenditure.

The software running on the electronic device 52 is preferably adapted to receive RMR data at intervals. Intervals may be frequent (such as an RMR measurement daily or every 2-3 days) during the early stages of the program, and less frequent (such as an RMR measurement every week, 2-3 weeks, or monthly) during later stages when metabolic changes would be expected to be smaller over a given time period. The software may be used to prompt the user to measure RMR, based on the stage of the weight control program, changes in other monitored parameters, and previous RMR changes. The time intervals between RMR measurements may be increased in length after an initial period has elapsed. For example, if RMR changes are expected (for example, from studies) to occur largely in an initial two week period of a health monitoring program, in which the user's caloric intake is initially reduced a certain percentage, then RMR measurements may be made every two days during this initial period, during which goals may be revised according to the actual RMR change. For example, a greater weight loss goal may be suggested if RMR does not fall, or increases due to activity. The initial period (for frequent RMR testing) may be restarted if caloric intake is modified significantly. However, if RMR settles to a stable level during the initial period, then the time intervals between RMR determination may be increased, e.g. to biweekly, after the initial period has elapsed. Intervals may also be adjusted continuously based on the actual changes in RMR, for example the next RMR measurement may be scheduled at a future time at which RMR may be predicted to have fallen by some value (perhaps defaulting to a maximum time interval if RMR is stable).

The health and fitness of the user can be monitored by monitoring some combination of body weight, RMR, body fat percentage, and other physiological parameters. The present invention provides a method of managing the health of a user by determining the resting metabolism of the user using an indirect calorimeter at suitable intervals; recording data indicative of foods consumed by the user over time; recording data indicative of activities performed by the user over time; processing the food data to determine caloric intake; and processing the activity data and the resting metabolism to determine caloric expenditure. Hence a caloric balance can be determined for the user, as described above. Other physiological parameters may be monitored in conjunction with diet and activity levels. Processing steps are preferably carried out using the computing device 52, but other devices in communication with device 52 may also provide additional data and processing support.

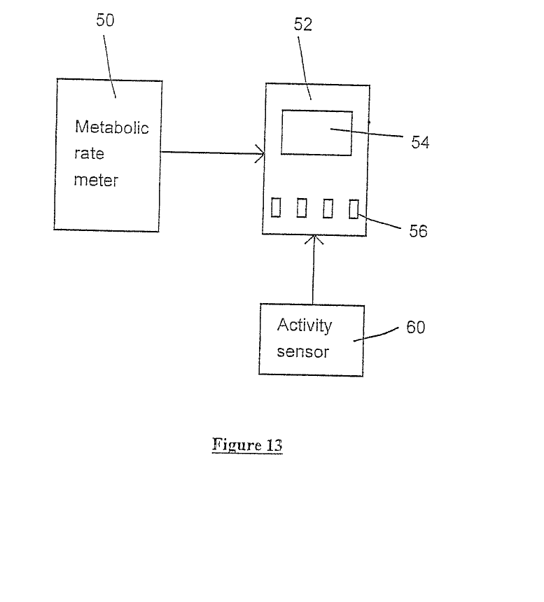

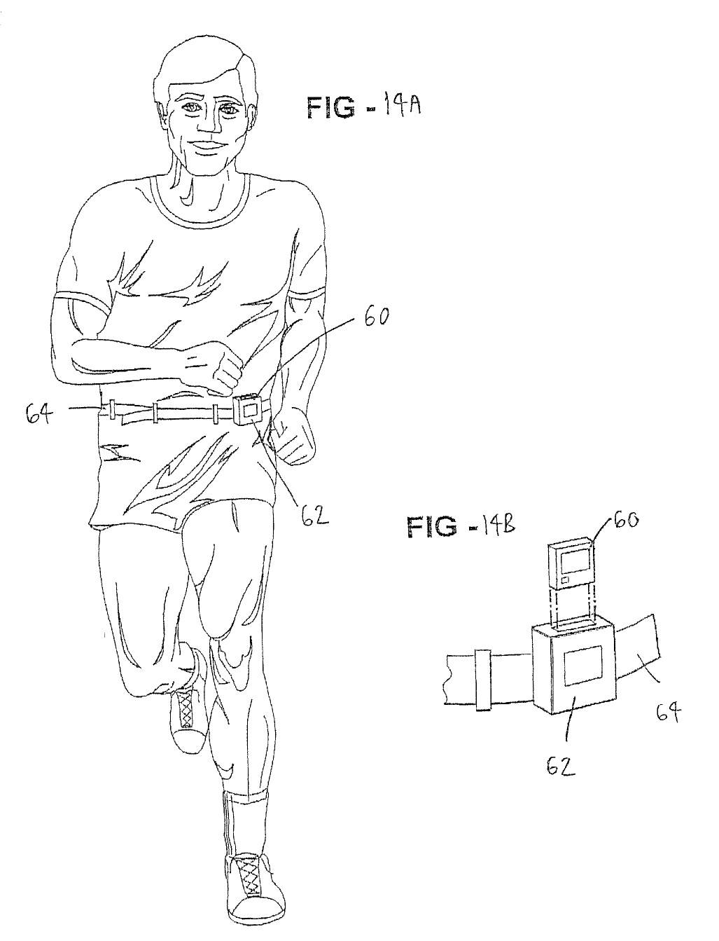

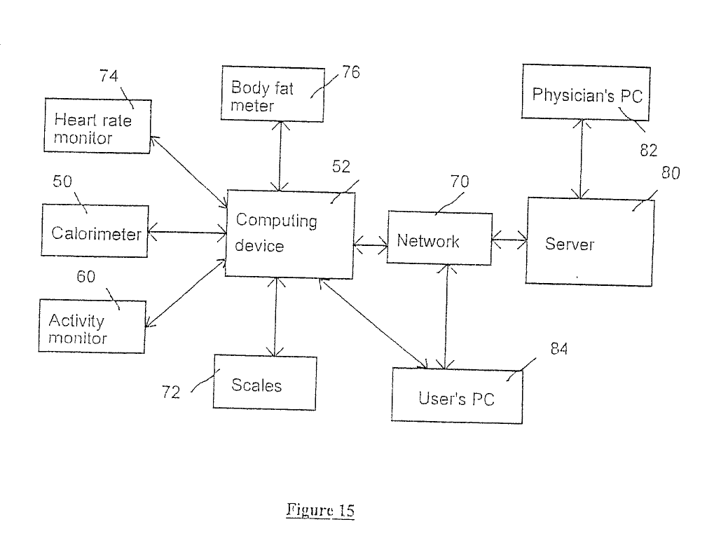

FIG. 13 shows another embodiment of the present invention. As described above, computing device 52 receives data at intervals from indirect calorimeter 50, relating to the resting metabolic rate of the user. The computing device also receives physical activity data from activity sensor 60 regarding the user. Preferably, this is a body or clothing mounted accelerometer, providing a signal related to physical activity. Such devices are well known to those skilled in the exercise arts. Accelerometers may provide a signal related to subject movement along one or more axes. The signal may be correlated with an increased metabolic rate associated with the physical activity by calibrating the sensor 60 with an indirect calorimeter. This has been described in co-pending U.S. Ser. No. 09/684,440, which is herein incorporated by reference in its entirety.

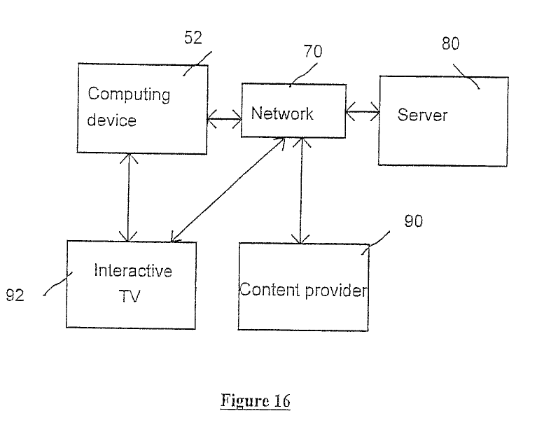

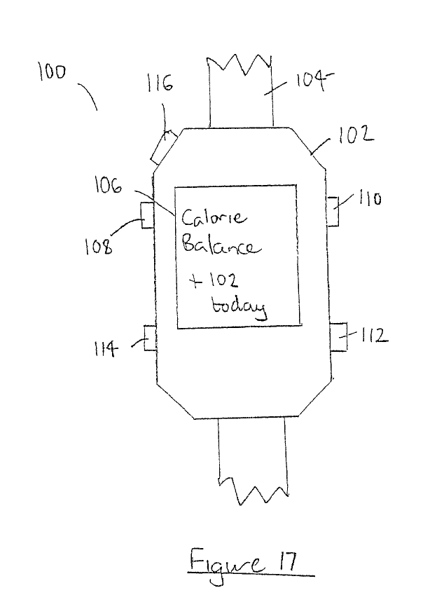

FIGS. 14A and 14B show an activity sensor 60 carried in a holder 62 held on belt 64 around the user. The activity sensor may be in the form factor of a module which is plugged into the computing device 52, which gives the device 52 the functionality of a pedometer or other activity sensor. This has been described more fully in pending U.S. patent application Ser. No. 09/669,125, herein incorporated in its entirety by reference. For example, in the case that device 52 is a PDA, the activity sensor 60 may be in the form of a module, harness, frame, or card which plugs into, docks with, or otherwise interfaces with a PDA and provides the PDA with an additional pedometer function.

The activity module may be clipped onto clothing, a belt, strap, or be adhered to the user's body. The activity module may display data which can be entered manually into device 52, such as a number related to cumulative activity. In a preferred embodiment, device 60 transmits activity related data to device 52 using a wireless link such as the Bluetooth protocol, or an IR method. A cable or other interface may also be used.

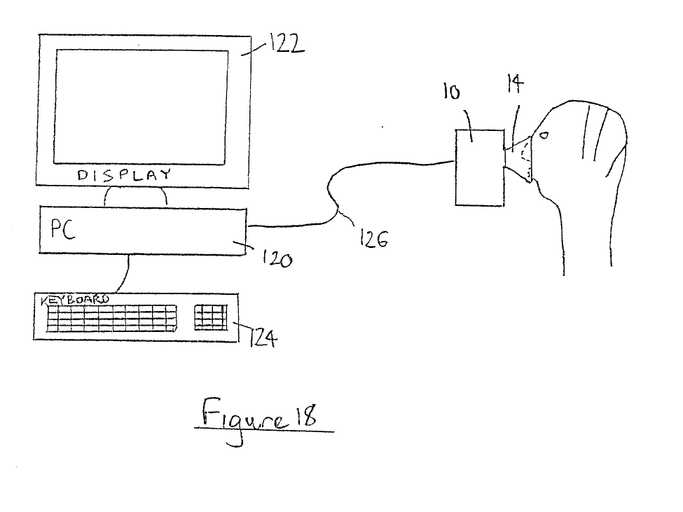

FIG. 15 illustrates another system embodiment of the present invention. Computing device 52 receives data at intervals from indirect calorimeter 50. Device 52 also receives data from activity sensor 60 to determine the level of physical activity of the user. A heart rate monitor 74 may also be used. The user's weight, determined by scales 72, is also entered into the computing device at intervals. The body fat percentage of the user is determined at intervals using body fat meter 76. The scale 72 and body fat meter 76 may be combined into a single device such as scales providing electrodes for bioimpedance measurements, such as those available commercially from Tanita and other companies.

Alternatively, body fat meter 68 and computing device 52 may be combined into a single device, or the body fat meter 68 may be an accessory module to computing device 52. For example, computing device 52 may provide electrodes and measurement circuitry so as to determine body fat using bioimpedance. This has been described more fully in pending U.S. provisional application Serial No. 60/219,069, filed Jul. 18, 2000, herein incorporated in its entirety by reference. This and other physiological monitors have been described more fully in pending U.S. patent application Ser. No. 09/669,125, incorporated herein in its entirety by reference.

Caloric data is received by health management software on computing device 52. Device 52 may be connected to a communications network 70, such as the Internet. In the embodiment that computing device 52 is a PDA, the PDA preferably has a wireless connection to the Internet. The PDA may also be docked or otherwise brought into communications with another device having a link to the communications network. For example, the PDA may be docked with a desktop personal computer having Internet access.

Data collected by device 52, related to the health and weight status of the user, may be transmitted via communications network 70 to a remote computer system (for example, a server system) 80. Remote computer system 80 comprises memory for storing information related to the user on a database. Remote system 80 may also have software for provision of feedback to the user. For example, a computer expert system may be used to provide feedback to the user. The user or other authorized person may access information on the database related to the user, for example through an Internet website. For example, information related to the user may be accessed by a physician, dietician, nutritionist, fitness adviser, physician, other health professional, or other lifestyle expert. A physician may use a personal computer 82 linked to the remote computer system 80 (possibly through communications network 70). In another example, a nutritionist may access the database of foods consumed by the user and weight trends, and provide feedback to the user in terms of foods to avoid and alternatives to previously consumed items of poor nutritional value. A weight control or health management business may provide computing devices such as 52 to multiple users, and have the multiple users communicate data relating to their health or weight management programs to one or more remote computer systems (such as Web servers), so that an employee of the business or other authorized person may access data of multiple users.

A weight control business may provide personal digital assistants, or software customized to run on personal digital assistants, to a large number of consumers. The weight loss business may provide an interactive website accessible through a communications network such as the Internet. The website may be used by the consumers for the storage, display, and analysis of data collected. The collected data may also be used to monitor trends amongst the consumer base, hence enabling the improvement of advice given to any individual consumer.

The user may carry or otherwise interact with one or more physiological monitors. Physiological parameters and monitors may include heart rate (for example using sensor 66), respiration rate, electrocardiograms, body temperature, and other parameters.

FIG. 16 illustrates another system embodiment by which feedback is provided to the user. Computing device 52 is used to collect information regarding the user, such as metabolic rate, diet log, activity levels, and physiological parameters, as described above. We will refer to this collectively as lifestyle information. Lifestyle information is stored within a database on remote computer system (for example, a server system) 80. Lifestyle data is transmitted from computing device 52 to computer system 80 through communications network 70, preferably the Internet. The computer system 80 analyzes the lifestyle data and determines appropriate feedback. The feedback is provided either by computer system 80, or by another feedback provider 90.

The user may view feedback on the display of computing device 52, but preferably views feedback on an entertainment device 92. Examples of an entertainment device include an interactive television, to be described, such as a personal computer, web access device, web TV, or other such audio-visual entertainment device.

The computing device 52 may also be used to transmit weight control related data to the interactive television or other device connected to communications network 70 by any convenient means. The Bluetooth protocol may be used for all short range communications and data transfer described in this specification. IR beams, cables, optical methods, memory module transfer, electrical interfaces, and ultrasound may also be used. In embodiments in which the computing device 52 is a PDA or other handheld device, it may also be used as a remote control to control entertainment devices.

For example, suppose information provided by an indirect calorimeter indicates that the user's metabolic rate has fallen during the weight control program. It would be advantageous for the user to engage in enhanced levels of physical activity in order to increase their resting metabolic rate. Based on the user's demographic data (age, gender), weight, and previous levels of activity, an exercise program can be devised for the user. A video program may be compiled from various appropriate segments and viewed by the user on the entertainment device 92.

The format and style of the feedback may be varied to optimize the response of the user. For example, the format might be in a news style, containing phrases such as “In breaking news, doctors have shown that increased exercise leads to enhanced resting metabolism and diet success.” The style and tone of the feedback may be matched to an optimum response of the user using the results of previous testing, questioning, previous success or failure at weight control, or other information regarding the user. For example, the feedback may be humorous, serious, nagging, etc. An authority figure, such as a simulation of the president, may be used to provide feedback to the user.

The remote server 80 preferably has an application program for receiving, storing, displaying and analyzing the information from the PDA relative to the user's physiological status, activities, and consumption. The information may be transmitted at intervals to healthcare professionals overseeing the weight loss program such as nutritionists, physicians and the like. Based on communications from the healthcare professionals to the website, and/or analysis performed on the website, messages could be transmitted by the server via the communications network to the PDA for display by the PDA to the patient. The messages could deal with the patient's program and could include messages as to modifications in the patient's conduct, including tests to be conducted or intervals for such tests, and information related to food consumption. The messages may include encouragement or criticism of past results. The system provides regular oversight which is highly successful in other commercial weight loss programs. The patient's response is enhanced by the knowledge that their progress will be communicated either to a health professional or to a computer program overseeing their progress. The computing device 52 may also receive information on the user's state of mind, for example relative to their happiness with the plan's diet and feelings of success of the program.

Preferably, the user records food and beverage consumption on the computing device 52 immediately after consumption. However, informal records may be stored on the computing device, such as voice records, image records, notes, data from barcode scanning, data from optical character recognition scanning, and used at a future time to create a formal diet log. A formal diet log may be created by the user or by other authorized persons with access to the data. For example, a weight control business may provide employees to create a formal diet log from informal records captured by the user of the system.

Electrical signals sensed by physiological monitors may be transferred to the computing device 52 through either wired or RF or other wireless links. The physiological monitors could incorporate connectors to receive removable memory sticks or cards such as flash memory or battery supported memory. These sticks or cards could be connected to the physiological monitors during monitoring and later removed and inserted into the PDA. The same memory module may be used with a variety of physiological monitors or employing a common data format.

Data collected by a portable computing device may be transferred to a portable device carried by a healthcare professional, such as a nutritionist, allowing the nutritionist to review the data on their own device and hence provide improved feedback and advice to the user.

FIG. 17 illustrates still another embodiment of the present invention of a wrist-mounted device for the computing device 52. This is described more fully in pending U.S. Ser. No. 09/745,373, the contents of which are incorporated in its entirety by reference. A person wears the wrist-mounted device, shown generally at 100, which resembles a watch. The device has main housing 102, and strap 104 to place around the user's wrist. A display 106 is used to show time, caloric balance, a diet input menu screen, an activity input menu screen, and an RMR input screen. A mode button 108 is used to change display mode. Buttons 110, 112, and 114 may be used to navigate through menu option choices, and select data items to record. The caloric balance for a person is related to the caloric intake compared with the caloric expenditure as previously described. Barcodes on prepackaged foods may be read by a barcode reader 116 associated with the housing 102 of the wrist-mounted device, and barcode data converted to nutrition data using a database. If a person is eating prepackaged foods from a limited selection, for example, meals supplied as part of a weight control program, the database relating barcode data to nutrition information may be conveniently stored within memory within the housing of the wrist-mounted device. An enhanced database may be stored on a remote server in communication with the wrist-mounted device through a communications network.

Access to an indirect calorimeter may be provided at a physician's office, nutritionist's office, fitness center, retail center, and the like. FIG. 18 shows a configuration suitable for use at a fitness center. FIG. 18 shows a user breathing through mask 14 of indirect calorimeter 10, which is in electrical communication with a desktop computer 120 provided by the fitness center. The fitness center may provide calorimeter 10, or the user may provide the calorimeter or disposable elements such as mask 14. The user, a customer of the fitness center, enters login information into the personal computer and breathes through the indirect calorimeter so as to measure their metabolic rate and store this in a computer system belonging to the fitness center, possibly a remote server in communication with PC 122. The fitness center may maintain a health and fitness database related to the user, containing data such as weight, RMR, diet information, and exercise performed. The fitness center may provide advice and feedback regarding the user's progress towards weight goals and fitness goals. An increased metabolic rate is indicative of an increased level of muscle mass, or reduced fat percentage for a constant body weight, hence is a desirable goal for users of the fitness center. The processor from the previous described embodiment of the indirect calorimeter (U.S. patent application Ser. No. 90/630,398) may be removed and signals from the transducers within the flow path of the indirect calorimeter used to send signals to a separate module which may be placed between the indirect calorimeter and personal computer. This reduces the weight of the device placed on the user's face, and also reduces sources of heat near the flow path which may reduce the accuracy of gas flow sensors. Data collected by exercise machines located within the fitness center may also be added to a database regarding the user stored on the computers of the fitness center.

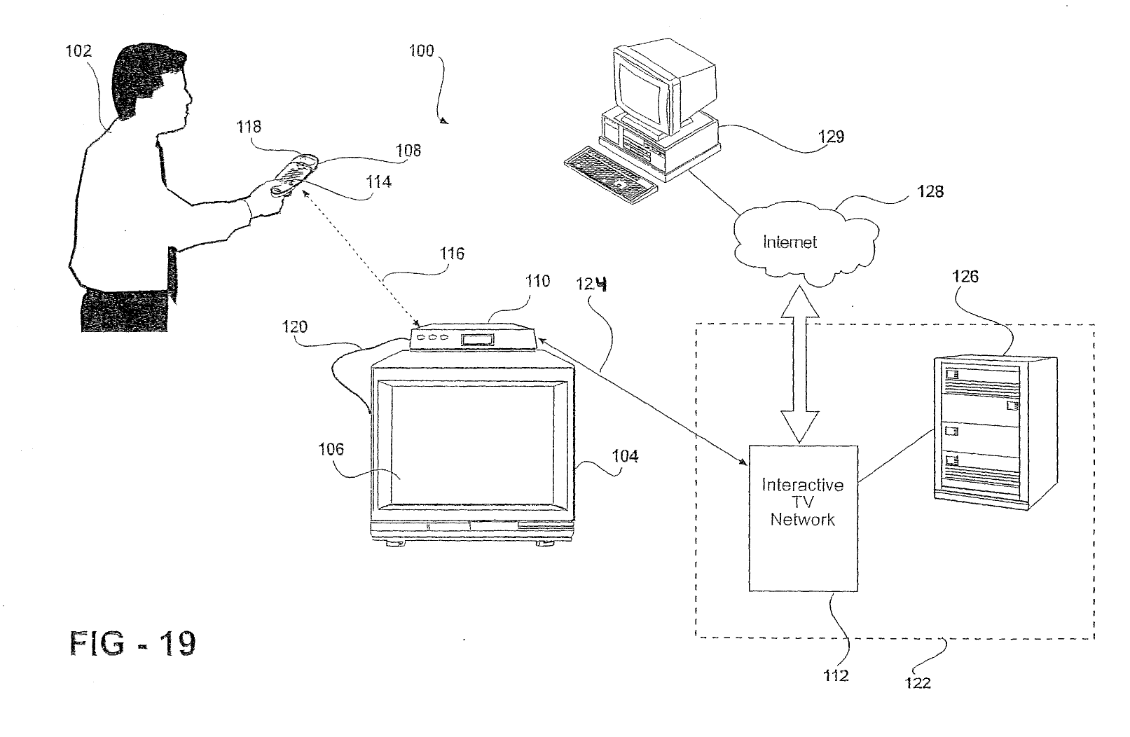

Referring to FIG. 19, a further embodiment of a system 100 for use in implementing the previously described method of integrated calorie management is provided. Advantageously, the system 100 incorporates the benefits of interactive television as part of a weight management program also referred to as a weight control program. The system 100 includes a user 102 having access to an entertainment device 104, such as a television. The television 104 is known and conventional in the art, and includes a screen 106 for viewing a visual portion of a signal and speakers (not shown) for hearing an audio portion of the signal.

The system 100 also includes a remote control device 108 for communicating with the television set 104 through a control means 110. It is contemplated that the control means 110 is a microprocessor or the like for receiving, processing and transmitting a signal. The remote control device 108 controls predetermined functions related to the operation of the television 104, such as turning the television 104 on or off, adjusting a channel or the volume, or the like. In addition, the remote control device 108 provides for interactive communication between the user 102 and an interactive television network 112, to be described.

The remote control device 108 includes an input mechanism 114 for communicating the user's intent to the control means 110 via an input signal. Preferably, the input mechanism 114 is a keypad button, toggle, joystick or the like used to transmit a signal representing a desired action to the control means. The input signal is transmitted between the remote control device 108 and the control means 110 via a telecommunications link 116. The telecommunications link 116 can be a wire operatively connecting the remote control device 108 and the control means 110. Alternatively, the telecommunications link is wireless link. One example of a wireless link is an infrared signal. Another example of a wireless link is a universal short wave wireless connectivity protocol referred to as Bluetooth, as is known in the art. Still another example of a wireless link is a memory module, also known as a memory stick. It is also contemplated that the remote control device 108 may include a display screen 118 that provides visual information. Alternatively, the display screen 118 is a touch sensitive screen, as another input mechanism 114. The remote control device 108 further includes other components, such as a signal processor, a transmitter, receiver or the like to operatively carry out its functions, as is understood in the art.

The control means 110, also referred to as a set top box, is a “smart” box providing a communication link between the user 102, the television 104 and an interactive television network 112. The control means 110 includes a signal receiver that receives information from an input source such as the remote control device 108, or the interactive television network 112, a processor that processes the information, and a transmitter that transmits the information to the television 104 in the appropriate format. The set top box 110 is operatively connected to an input means (not shown) on the television 104, such as via a cable 120. The set top box 110 sends a signal to the television set 104 via the cable 120 to control operation of the television set 104, such as volume, tuner and on-screen display. The television set 104 interprets the information and responds accordingly. For example, the user 102 selects a channel and the television set 104 displays the appropriate image on the screen 106 and broadcasts the desired sound through its speakers. In another example, the television volume is modified either to increase or decrease. In still another example, the television set 104 is turned on or off.

The set top box 110 is in communication with the interactive TV network 112 via a communications network 122. The communications network 122 includes a central programming network that receives information from a number of sources and distributes the information to local programming providers. As is known in the art, the signal is transmitted via a communication link 124 between the central network and local provider through a network of hubs and nodes. Various types of communication links 124 are conventional and known in the art to facilitate the transfer of information within a communications network. One example of a communication link is a wired connection, such as a fiber optic cable. The cable communication link facilitates communication between the central programming network, the local provider, and the user. Another example of a communications link is a satellite signal transmitted between the central programming network and the local provider, or the local provider and a user satellite receiver mechanism. It is contemplated that the communication link between the user satellite receiver mechanism and set top box 110 is a wired connection.

The interactive TV network 112 is part of the communications network 122, and is a provider such as a central programming network or a local provider. The interactive TV network 112 includes a computer system having a memory, a database 126 stored in the memory and a processor. Preferably, information regarding the method of integrated calorie management is maintained in the database 126. The computer system interactively communicates with the user 102 through the set top box 110, via the communications network 122. Alternatively, information regarding the method of integrated calorie management is maintained on another computer system 129 that communicates with the interactive television network computer system via an internet, and in particular the Internet 128. The Internet 128 is a global system of interconnected networks that utilize a standard Internet Protocol (IP) as the network layer protocol. The Internet 128, also referred to as the World Wide Web, is an accepted gateway for the transfer of information, through the use of hypermedia.

In operation, the user 102 uses the input mechanism 114 of the remote control device 108 to send a signal to the interactive TV network 112 via the set top box 110 to access the balance log weight control program. The set top box 110 processes the signal, and displays a window for the balance log on the television set 104. The television set 104 is used as a display monitor, to display a series of windows, referred to as screens. Preferably, the first page presented to the user 102 is a home page consisting of a main page presenting an attractive overview of the available information, with more specific information embedded in subsequent pages. Advantageously, the user 102 can simultaneously view television programming and the balance log pages. Advantageously, the user 102 can make choices from options displayed in a window on the television screen 106 using the remote control device 108. For example, the user 102 can scroll through a menu, point to or highlight an option, and select that option by manipulating the input mechanism 114 on the remote control device 108. The information displayed on the television screen 106 is provided to the television set 104 by the interactive television network 112 via the communications network 122. The set top box 110 processes the signal containing instructions from the remote control device 108 and transmits the processed signal to the interactive television network 112. The interactive television network 112 uses the instructions and updates the balance log accordingly. Advantageously, the user 102 can interact with the interactive television network 112 on a real-time basis.

The system 100 may include other components, such as the computing device 52 previously described or the like to carry out the method of integrated calorie management, to be described.

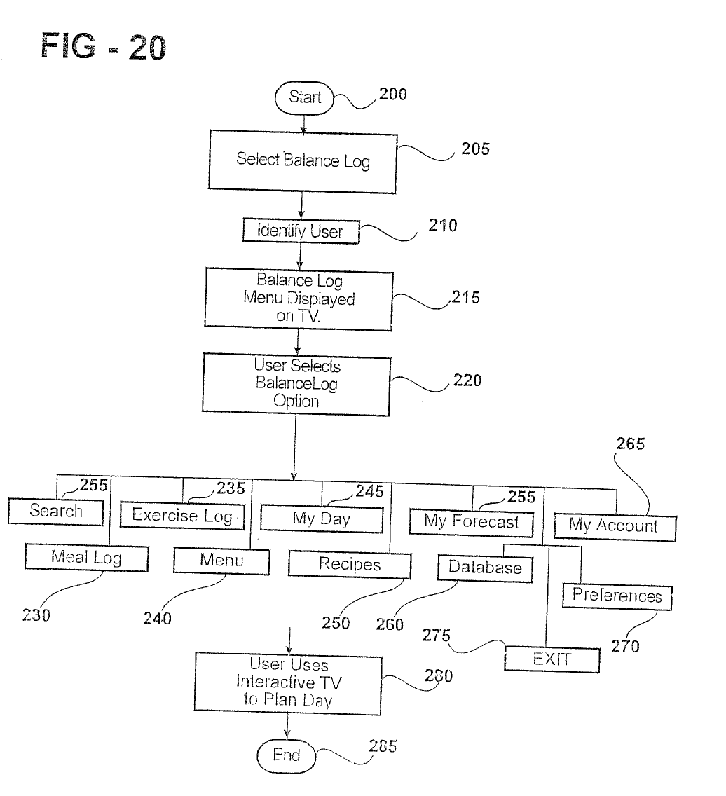

The method of integrated calorie management using interactive television is illustrated in FIG. 20. The method is implement by the system 100, as previously described. The methodology begins in block 200 when called for by the user 12, and advances to block 205. In block 205, the user 12 selects the balance log weight management option from a menu displayed on the screen 106 of the television set 104. Preferably, the user uses the remote control device 108 to send a signal to the interactive television network 112 via the set top box 110 to display the menu. Alternatively, the user 102 activates a control, such as a button integral with the set top box 110, to communicate with the interactive television network 112.

As previously described with respect to FIGS. 4-12, the balance log weight management program correlates resting metabolism with calorie intake and activity level to provide the user 102 with a caloric balance for use as part of a weight management program. The methodology advances to block 210.

In block 210, the user 102 is identified by the interactive TV network 112 as a participant in the balance log weight management program. For example, the user 102 may be requested to provide a password or other such identification using the remote control device 108. The interactive TV network 112 checks the password against information in its database 126 to determine if user access is permitted. If user access is permitted, the methodology advances to block 215.

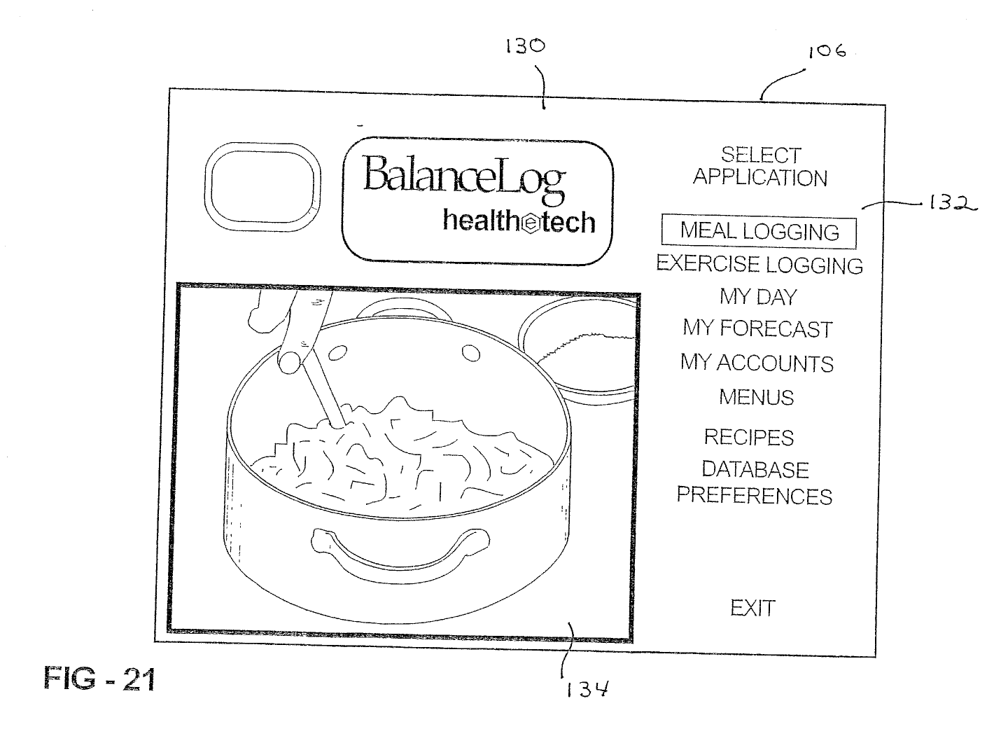

In block 215, the methodology presents the user 102 with a window 130 or page displayed on the television screen 106 for the balance log weight management program containing transactional selections for using the program. It should be appreciated that the balance log page can be displayed on the entire television screen 106, or on a portion of the screen 106. Preferably, the first window is a home page consisting of a main page, with more specific information embedded in subsequent pages, as shown in FIG. 21 at 132. Advantageously, a television program can be simultaneously displayed with the balance log menu as shown at 134, in which case the screen is split to accommodate both displays. The television program may be related to the balance log weight management program, such as nutrition, exercise or cooking programming. The methodology advances to block 220.

In block 220, the user 102 selects a balance log transaction using the remote control device 108, or directly using a button on the set top box 110 from the options displayed on the television screen 106. Examples of balance log options includes search, meal logging, exercise logging, menus, my day, recipes, my forecast, database, my account, preferences or exit. In addition, the user may be presented with hyperlinks to other related websites.

If the user 102 selects a search option, the methodology advances to block 225 and a search page is displayed on the television screen 106. The search screen provides the user 102 with options for conducting a search of available information within the balance log weight management program. Types of information include recipes, exercises, daily caloric input, caloric balance, or the like.

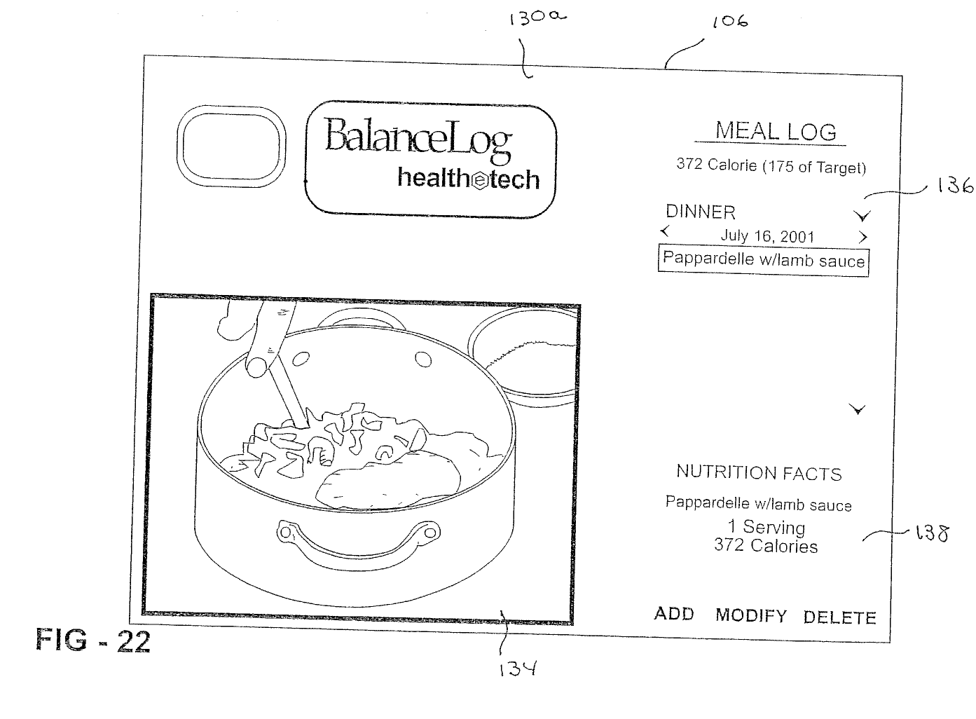

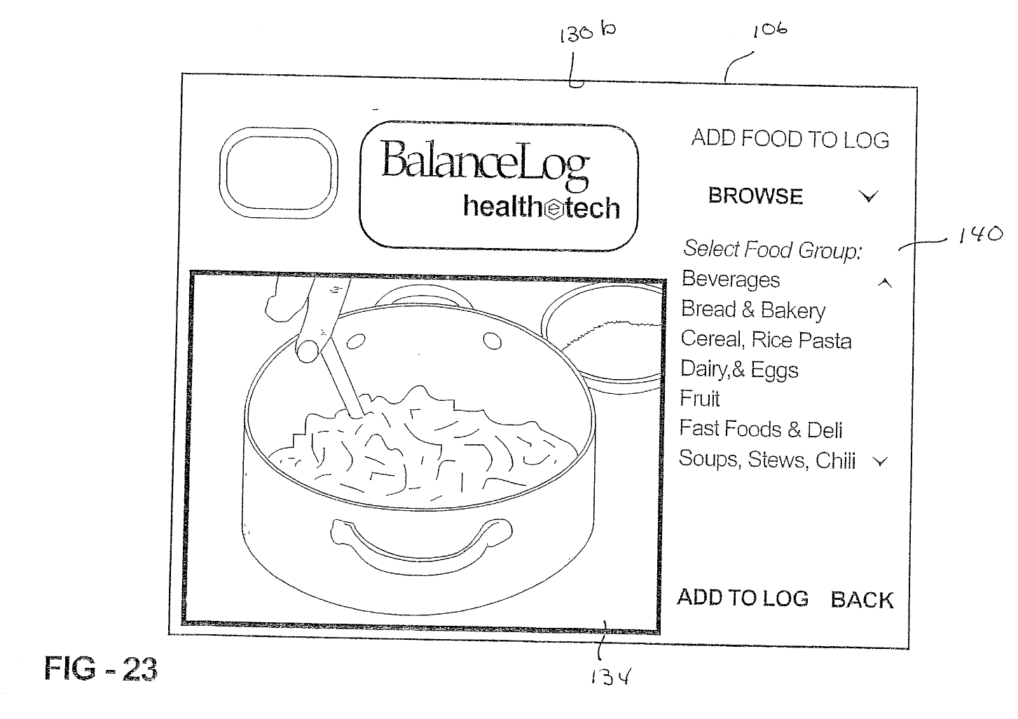

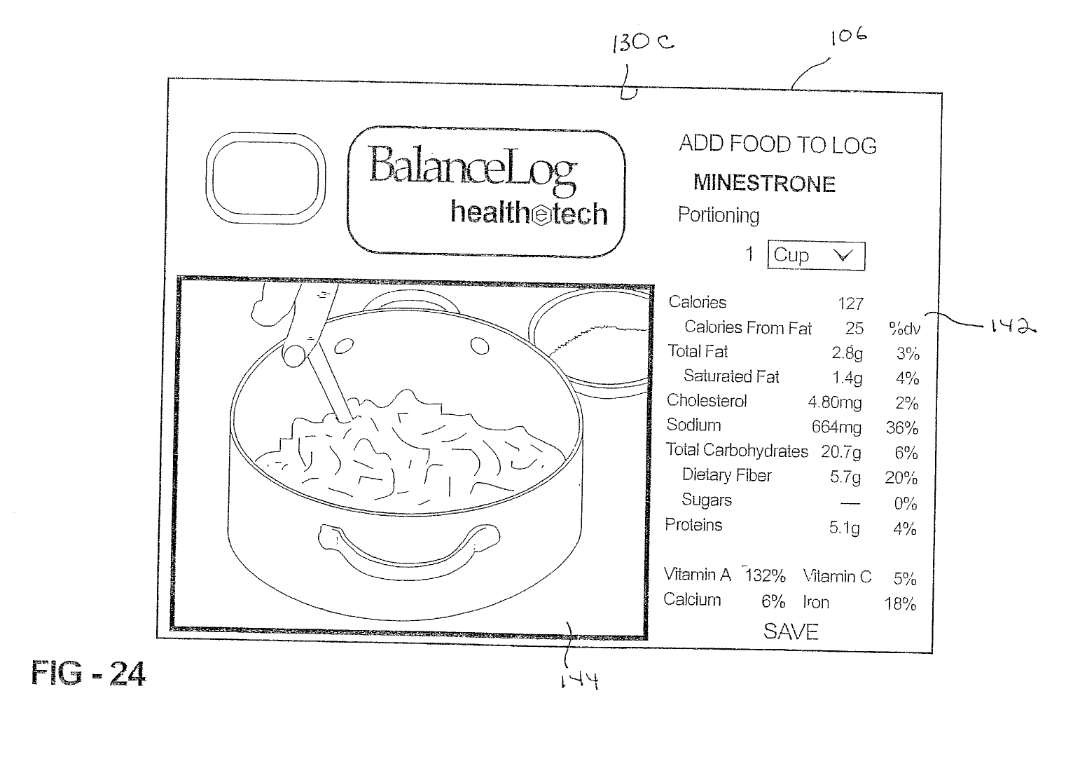

If the user 102 selects a meal log option, the methodology advances to block 230 and a meal log page 130a is displayed on the television screen 106, as shown in FIGS. 22-24. For example, the meal log option provides an opportunity for the user 102 to select meals for a predetermined time period, such as dinner, as shown at 136. The user 102 can select a meal entree from a predetermined list of entrees maintained in the database 126, and determine whether to prepare and eat the meal, depending on whether the meal fits their goals for the day. For example, the user 102 can use the meal log to plan their daily meals to ensure that the meals they consume correspond with their weight management goals. The user 102 selects a particular mealtime, such as dinner, and a dinner entree, and is provided with information such as nutritional value of the selected meal, includes caloric, fat and vitamin analysis as shown at 138. The user 102 is provided with other options, such as to search for a food from a page 130b listing a predetermined list of foods organized by food group, as shown in FIG. 23 at 140. The user 102 can add the selected food to the meal log, modify a meal log entree or delete an existing meal log entree. Advantageously, the user can view a page 130c listing the nutritional information associated with the food as shown in FIG. 24 at 142 and simultaneously view a television program on how to prepare the food as shown at 144.

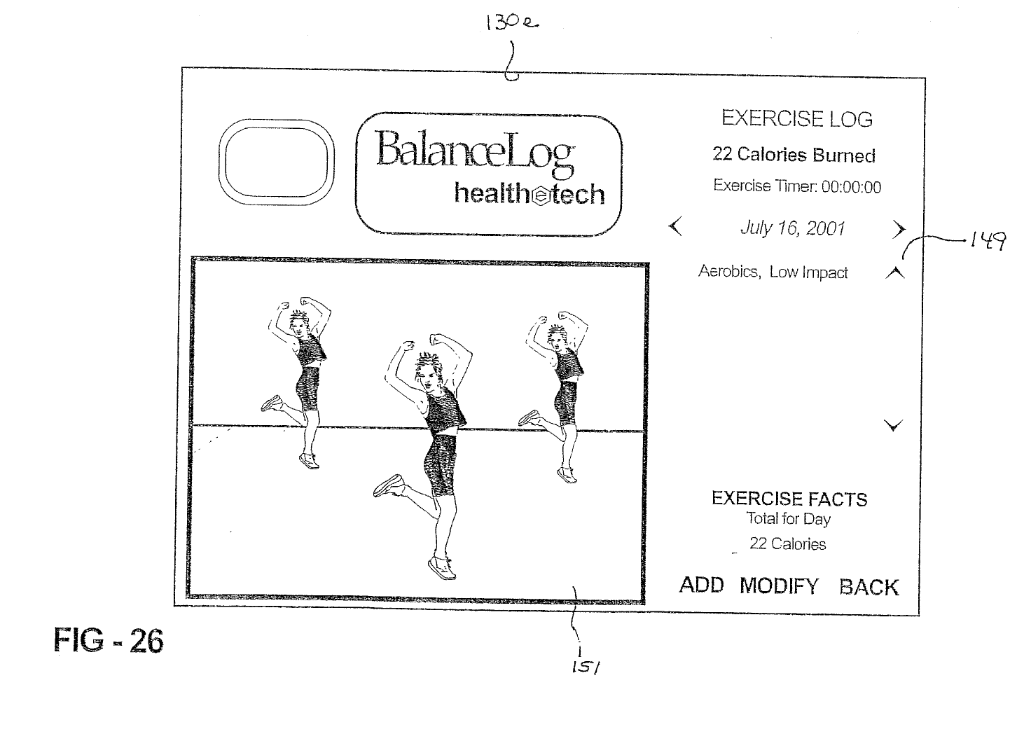

If the user selects an exercise log option, the methodology advances to block 235, and the user 102 is provided a page 130d listing exercise related information as shown at 146 in FIG. 125. For example, the user 102 can select an exercise using a search feature or from a predetermined list of exercises displayed on the screen 106. The user 102 can also select to learn more about the exercise, and view a video on how to perform the exercise as shown at 148. The user 102 can also learn facts about the exercise, such as the fat burning potential of the exercise. Alternatively, the user 102 can select an exercise as shown at 149 in FIG. 26 from a page 130e containing a dropdown menu, if they have performed that exercise that day, add the exercise to the balance log and indicate the exercise was performed and the amount of time spent performing the exercise. As shown in FIG. 26 at 151, the user 102 can select an exercise to interactively view on the television 104, and perform the exercise while simultaneously viewing it on the screen 106, and add the information regarding the performed exercise to the balance log weight control program.

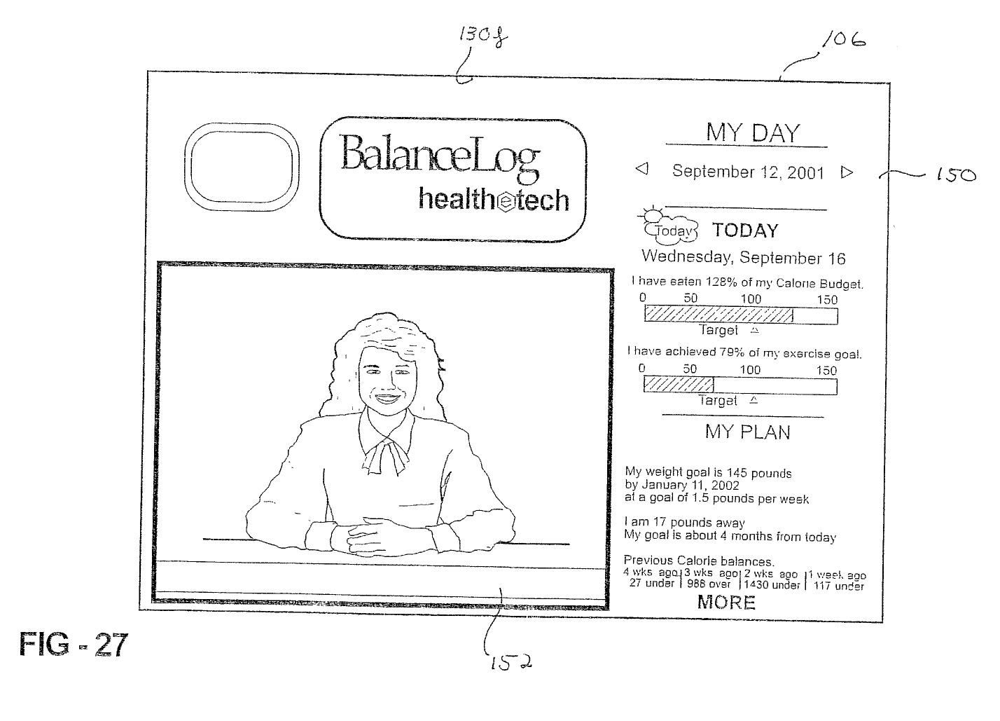

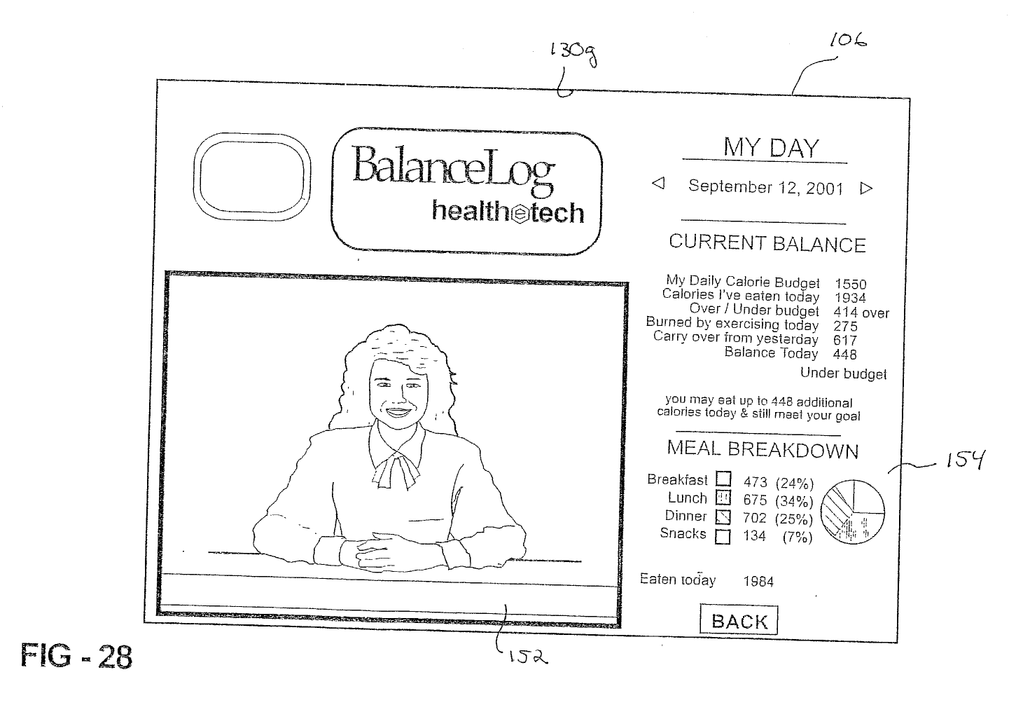

If the user selects a daily summary option, the methodology advances to block 245, and the user 102 is provided with a page 130f containing a summary of the information related to that particular day as shown in FIG. 27 at 150. For example, the user is provided with a summary of food consumed, percent of calorie budget for the day already eaten, percent of exercise goal reached, activity level, and caloric availability. In addition, the user 102 is presented with information regarding weight goals, and progress towards the weight goal for that day, or a previous day. Advantageously, the user 102 can access the balance log while viewing a program on the television set 104 as shown at 152. The balance log summary can also be presented on a page 130g in a graphical format, as shown in FIG. 28 at 154.

If the user 102 selects a my forecast option, the methodology advances to block 255 and the user 102 is presented with a page containing a summary of information relating to how well they are doing in attaining their weight management goal over a predetermined period of time.

If the user selects an account option, the methodology advances to block 265 and the user 102 is presented with a page containing a summary of information concerning their balance log weight management program. For example, the user 102 is presented with information such as personal details, including height, weight, age or other such information. The information may also include a summary of initial conditions, targets and goals, as previously described.

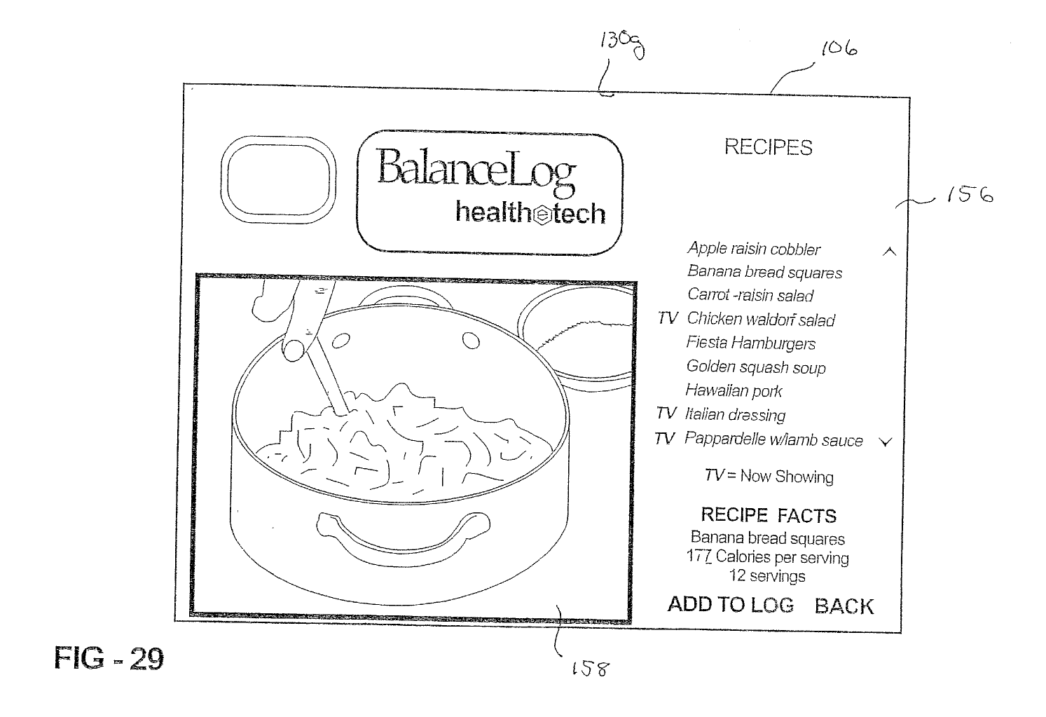

If the user 102 selects a recipe option, the methodology advances to block 250 and the user 102 is presented with a page 130g containing recipes as shown in FIG. 29 at 156.

Preferably, the recipes are maintained in a recipe database on the database 126. For example, the user 102 can search the recipe database for a particular recipe. Alternatively, the user 102 is presented with a page listing recipe titles, and the user 102 can select one recipe title to view more specific information regarding how to prepare the recipe. The user 102 may also have the option of adding the recipe to the user's balance log weight management program. The user 102 may interactively select to view a recipe being demonstrated on a program simultaneously displayed on the screen 106 of the television set 104 as shown at 158.

If the user 102 selects a database option, the methodology advances to block 260 and the user 102 is presented with a page on the interactive television relevant to the personal training program.

If the user 102 selects a preference option, the methodology advances to block 270 and the user 102 is presented with a page on the interactive television 104 that allows them to view, establish or modify user preferences. Preferably, the user preferences are taken into account by the personal fitness trainer in suggesting a personal fitness training program to the user. Examples of user preferences include type of food or exercise or lifestyle, specific health concerns or the like.

If the user 102 selects an exit option, the methodology advances to block 275 and balance log window is closed on the screen 106 of the television set 104.

Advancing to block 280, the user 102 uses the balance log weight management program, and more specifically the caloric balance from the balance log to integrate their calorie intake and their activity level within the weight management program. The methodology advances to block 285 and ends.

Returning to block 210, if the user 102 is not identified, the user 102 is provided an error message, and the methodology ends and the screen returns to television programming.

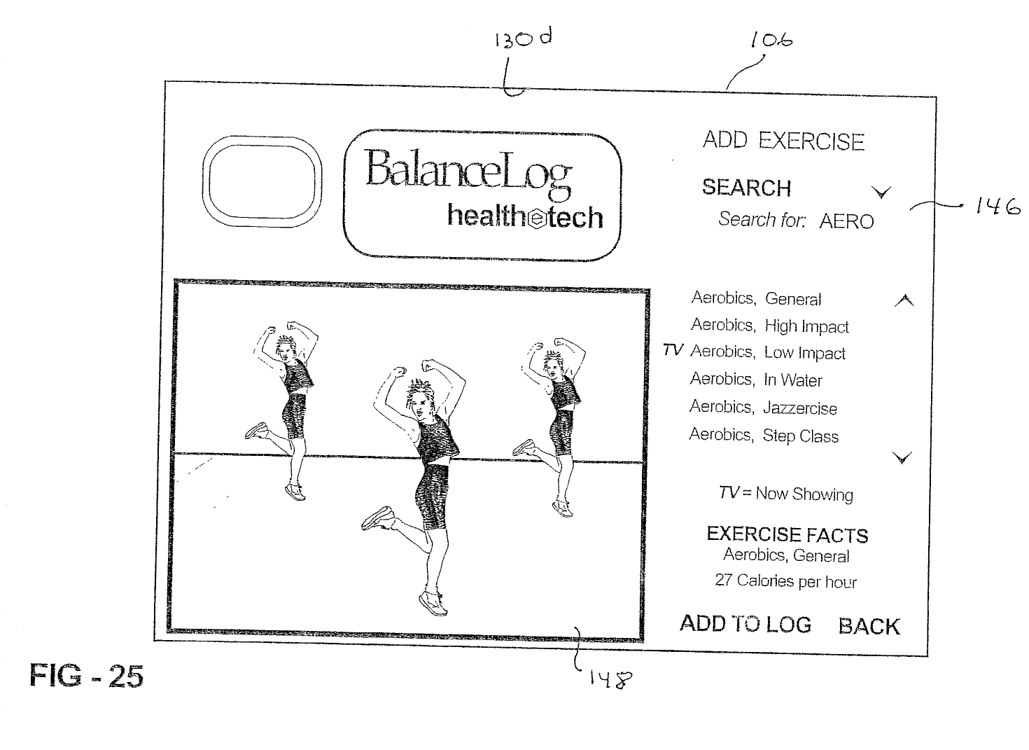

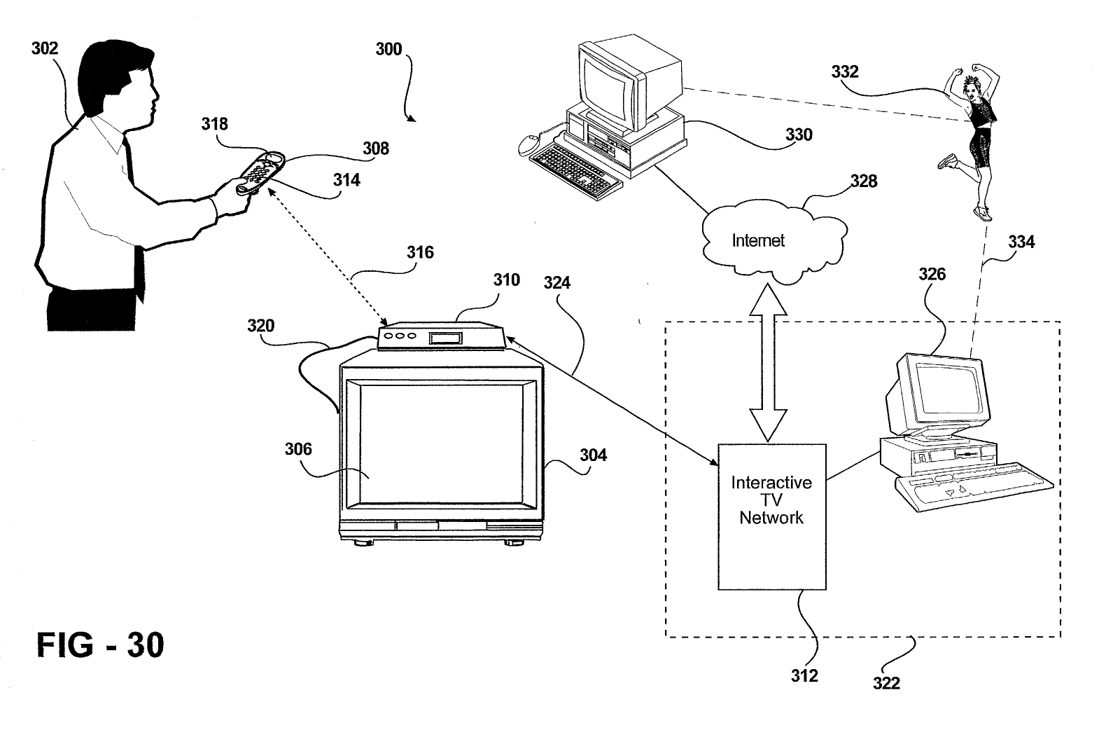

Referring to FIG. 30, a still a further embodiment of a system 300 for use in implementing an interactive television personal fitness training program as part of a health management program is provided. It should be appreciated that the system 300 can stand alone, or be integral with the health management program described with respect to FIGS. 1-29. Advantageously, the system 300 incorporates the benefits of interactive television and a personal fitness trainer into a personalized health management program. The system 300 includes a user 302 having access to an entertainment device 304, such as a television. The television 304 is known and conventional in the art, and includes a screen 306 for viewing a visual portion of a signal and speakers (not shown) for hearing an audio portion of the signal.

The system 300 also includes a remote control device 308 for communicating with the television set 304 through a control means 310. It is contemplated that the control means 310 is a microprocessor or the like for receiving, processing and transmitting a signal. The remote control device 308 controls predetermined functions related to the operation of the television 304, such as turning the television 304 on or off, adjusting a channel or the volume, or the like. In addition, the remote control device 308 provides for interactive communication between the user 302 and an interactive television network 312, to be described.

The remote control device 308 includes an input mechanism 314 for communicating the user's intent to the control means 310 via an input signal. Preferably, the input mechanism 314 is a keypad button, toggle, joystick or the like used to transmit a signal representing a desired action to the control means 310. The input signal is transmitted between the remote control device 308 and the control means 310 via a telecommunications link 316. The telecommunications link 316 can be a wire operatively connecting the remote control device 308 and the control means 310. Alternatively, the telecommunications link is wireless link. One example of a wireless link is an infrared signal. Another example of a wireless link is a universal short wave wireless connectivity protocol referred to as Bluetooth, as is known in the art. Still another example of a wireless link is a memory module, also known as a memory stick. It is also contemplated that the remote control device 308 may include a display screen 318 that provides visual information. Alternatively, the display screen 318 is a touch sensitive screen, as another type of input mechanism 314. The remote control device 308 further includes other components, such as a signal processor, a transmitter, receiver or the like to operatively carry out its functions, as is understood in the art.

The control means 310, also referred to as a set top box, is a “smart” box providing a communication link between the user 302, the television 304 and an interactive television network 312. The control means 310 includes a signal receiver that receives information from an input source such as the remote control device 308, or the interactive television network 312, a processor that processes the information, and a transmitter that transmits the information to the television 304 in the appropriate format. The set top box 310 is operatively connected to an input means (not shown) on the television 304, such as via a cable 320. The set top box 310 sends a signal to the television set 304 via the cable 320 to control operation of the television set 304, such as volume, tuner and on-screen display. The television set 304 interprets the information and responds accordingly. For example, the user 302 selects a channel and the television set 304 displays the appropriate image on the screen 306 and broadcasts the desired sound through its speakers. In another example, the television volume is modified either to increase or decrease. In still another example, the television set 304 is turned on or off.

The interactive television network 312 is part of a communications network 322. The communications network 322 includes a central programming network that receives information from a number of sources and distributes the information to local programming providers. It should be appreciated that the interactive television network 312 can be either a central programming network or a local programming provider. The set top box 310 is in communication with the interactive television network 312 via the communications network 322. As is known in the art, the signal is transmitted via a communication link 324 between the central network and local provider through a network of hubs and nodes. Various types of communication links 324 are conventional and known in the art to facilitate the transfer of information within a communications network. One example of a communication link 324 is a wired connection, such as a fiber optic cable. The cable communication link facilitates communication between the central programming network, the local provider, and the user. Another example of a communications link 324 is a satellite signal transmitted between the central programming network and the local provider, or the local provider and a user satellite receiver mechanism. It is contemplated that the communication link between the user satellite receiver mechanism and set top box 310 is a wired connection.

The interactive TV network 312 also includes a computer system 326 having a memory, a database stored in the memory, a processor and an input device such as a keyboard and a mouse. The interactive TV network computer system 326 communicates with a fitness trainer computer system 330 via an internet, and in particular the Internet 328. The Internet 328 is a global system of interconnected networks that utilize a standard Internet Protocol (IP) as the network layer protocol. The Internet 328, also referred to as the World Wide Web, is an accepted gateway for the transfer of information, through the use of hypermedia. The fitness trainer computer system 330 is accessed by a fitness trainer 332, and the personal fitness trainer 332 communicates with the user 302 via the Internet 328, and the communications network 322, which includes the interactive television network 312. Alternatively, the fitness trainer 332 uses the interactive television network computer system 326 to communicate with the user 302 via the communications network 322, as shown at 334.

In operation, the user 302 uses the input mechanism 314 for the remote control device 308 to send a signal to the interactive television network 312 via the set top box 310 to initiate the personal fitness training program. The set top box 310 processes the signal, and displays a window for the personal fitness training program on the screen 306 of the television set 304. The television set 304 is used as a display monitor, to display a series of windows, referred to as pages. Preferably, the first page presented to the user 302 is a home page consisting of a main page presenting an attractive overview of the available information, with more specific information embedded in subsequent pages. It is contemplated that the user 302 can simultaneously view television programming and the fitness training pages on the screen.

The user 302 makes selections from options displayed in a window on the television screen 306, and uses the remote control device 308 to communicate with the personal fitness trainer 332 via the interactive television network 312. For example, the user 302 can scroll through a menu, point to or highlight an option, and select that option by manipulating the input mechanism 314 on the remote control device 308. The information displayed on the television screen 306 is provided to the television set 304 by the interactive television network 312 via the communications network 322. The set top box 310 processes the signal containing instructions from the remote control device 308 and transmits the processed signal to the personal fitness trainer 332 via the interactive television network 312. In this manner, the user 302 interactively participates with the personal fitness trainer via the interactive television network 312 on a real-time basis, in a manner to be described.

The system 300 may include other components, such as the computing device 52 previously described or the like, to carry out the method of personal fitness training using interactive television, to be described.

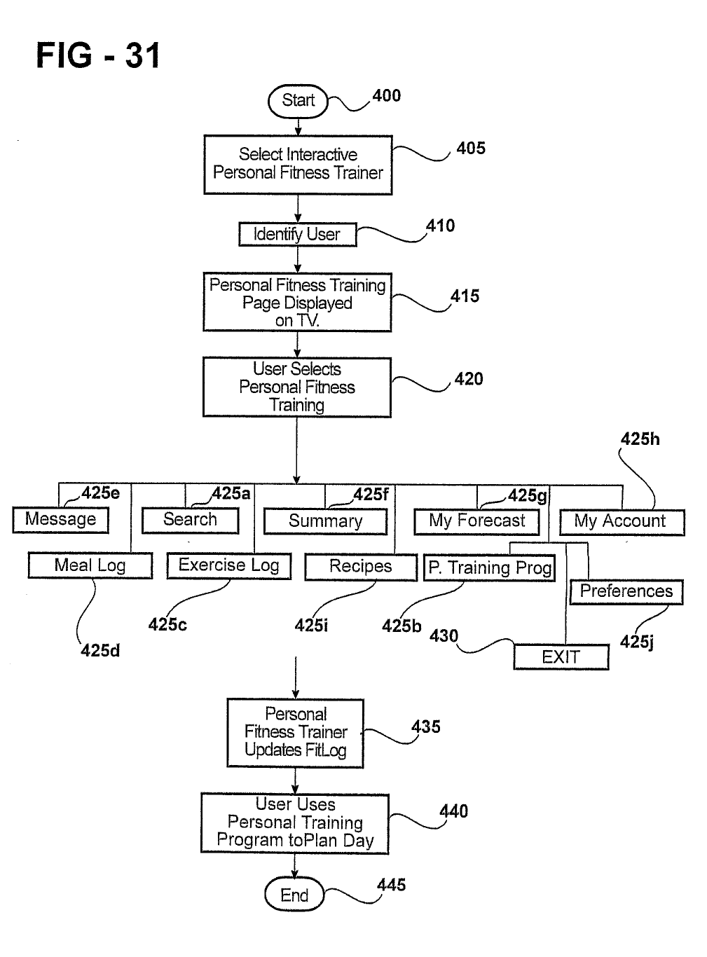

The method of personal fitness training using interactive television is illustrated in FIG. 31. The method is implemented by the system 300, as previously described. The methodology begins in block 400 when called for by the user 302, and advances to block 405. In block 405, the user 302 selects the interactive personal fitness trainer option from a menu displayed on the screen 306 of the television set 304. Preferably, the user 302 uses the remote control device 308 to send a signal to the interactive television network 312 via the set top box 310 to display the menu and make the selection. Alternatively, the user 302 activates a control, such as a button integral with the set top box 310, to communicate with the interactive television network 312.

It should be appreciated that the interactive television personal fitness training program, also referred to as FitLog, may be part of a balance log weight management program. As previously described with respect to FIGS. 1-30, the balance log weight management program correlates resting metabolism with calorie intake and activity level to provide the user 302 with a caloric balance for use as part of a health management program. The methodology advances to block 410.

In block 410, the user 302 is identified by the interactive TV network 312 as a participant in the interactive television personal fitness training program. For example, the user 302 may be requested to provide a password or other such identification using the remote control device 308. The interactive TV network 312 checks the password against information maintained in a database in its computer system 326 to determine if user access is permitted. If user access is permitted, the methodology advances to block 415.

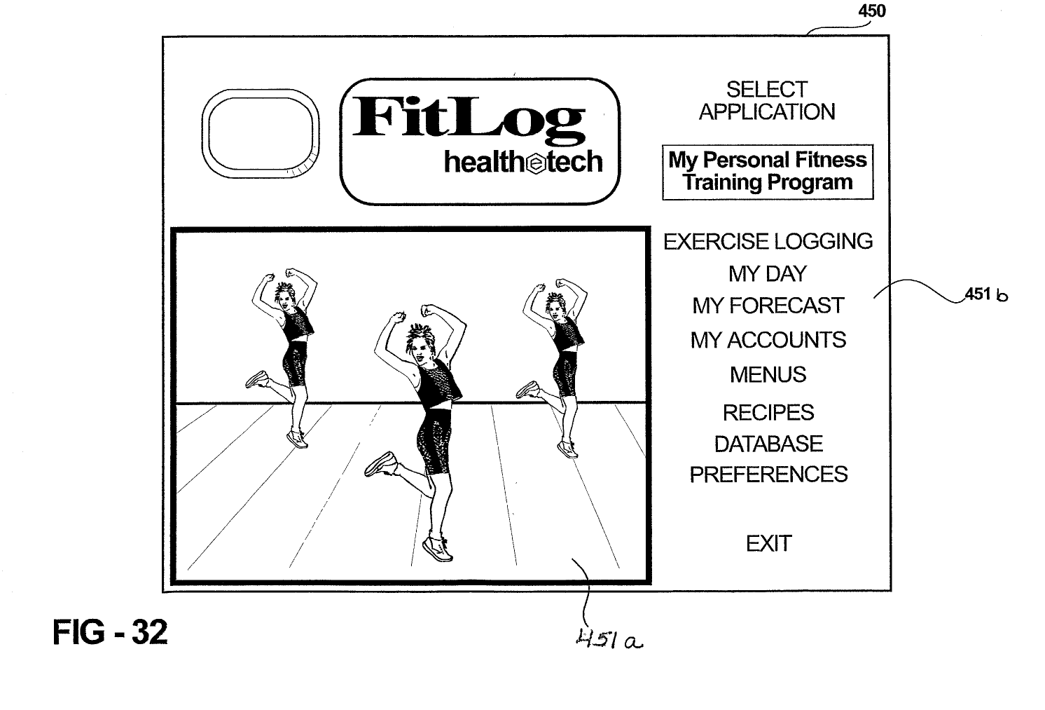

In block 415, the methodology presents the user 302 with a window or page displayed on the television screen 306 for the personal fitness training program containing transactional selections for participating in the program. It should be appreciated that the personal fitness training program page can be displayed on the entire television screen 306, or on a portion of the screen 306. Preferably, the first window is a home page consisting of a main page, as shown in FIG. 32 at 450, with more specific information embedded in subsequent pages. Advantageously, a television program 451a can be simultaneously displayed with the personal fitness training menu 451b, in which case the screen is split to accommodate both displays. The television program 451a may be related to the user's personal fitness training program, such as nutrition, exercise or cooking programming. The methodology advances to block 420.

In block 420, the user 302 selects a personal fitness training transaction using the remote control device 308, or directly using a button on the set top box 310 from the options displayed on the television screen 306. Examples of personal fitness training options includes “search,” “my personal fitness training program,” “meal logging,” “exercise logging,”“menus,” “my day,” “recipes,” “my forecast,” “my account,” “preferences” or “exit.” In addition, the user 302 may be presented with hyperlinks to other related websites.

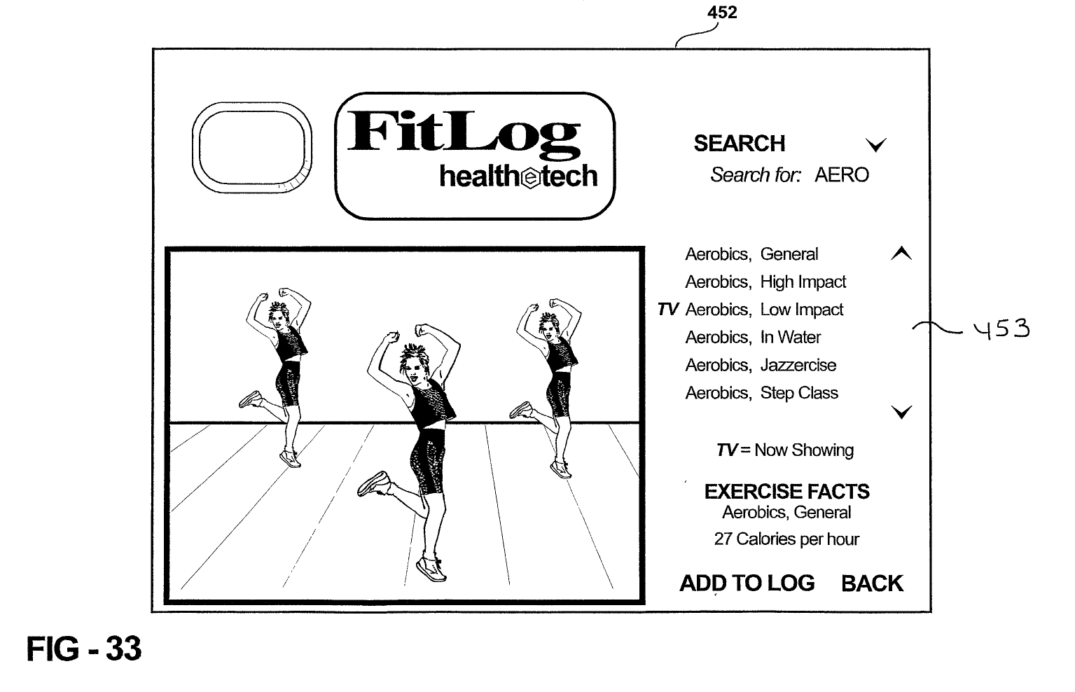

If the user 302 selects a search option, the methodology advances to block 425a and a search page is displayed on the television screen 306 as shown in FIG. 33 at 452. The search screen provides the user 302 with options shown at 453 for conducting a search of available information related to their personal fitness training program. Types of information include training schedule, exercises, daily caloric input, caloric balance, recipes or the like.

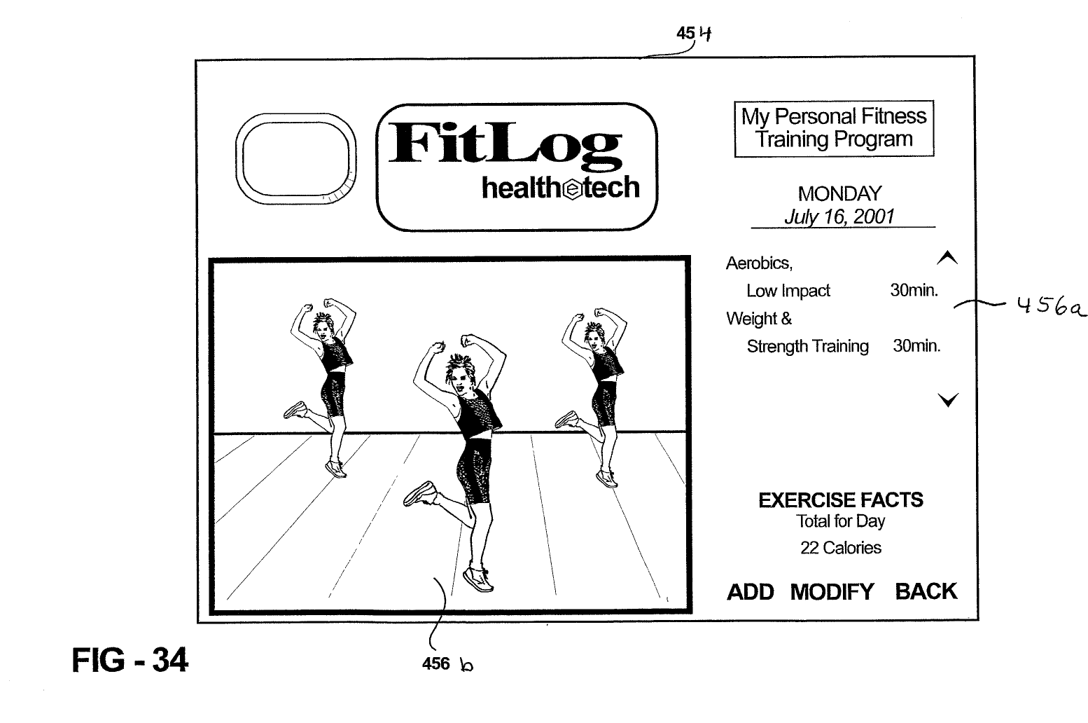

If the user selects the personal fitness training option, the methodology advances to block 425b, and the user 302 is provided a page containing a personal fitness program developed by the personal trainer specifically for the user, as shown at 454 in FIG. 34. The personal fitness program contains health related information specifically tailored for the user 302 by the personal fitness trainer 332.

For example, the personal fitness program prescribes exercises or activities for the user 302 to perform to meet a predetermined health management goal, as shown at 456a. The user 302 can also select to learn more about the exercise, and view a video on how to perform the exercise, as shown at 456b. It is contemplated that the user 302 can select an exercise to interactively view on the television 304, and perform the exercise while simultaneously viewing it on the screen 306. The user 302 can also learn facts about the exercise, such as the fat burning potential of the exercise.

If the user 302 selects the exercise log option, the methodology advances to block 425c. In block 425c, the user 302 is presented with a page containing a dropdown menu of exercise related activities. If they have performed an exercise related activity that day, the user can update their personal fitness training log to indicate the activity performed and the amount of time spent performing the activity. The user 302 is also able to update the fitness program with other information regarding the performed exercise.

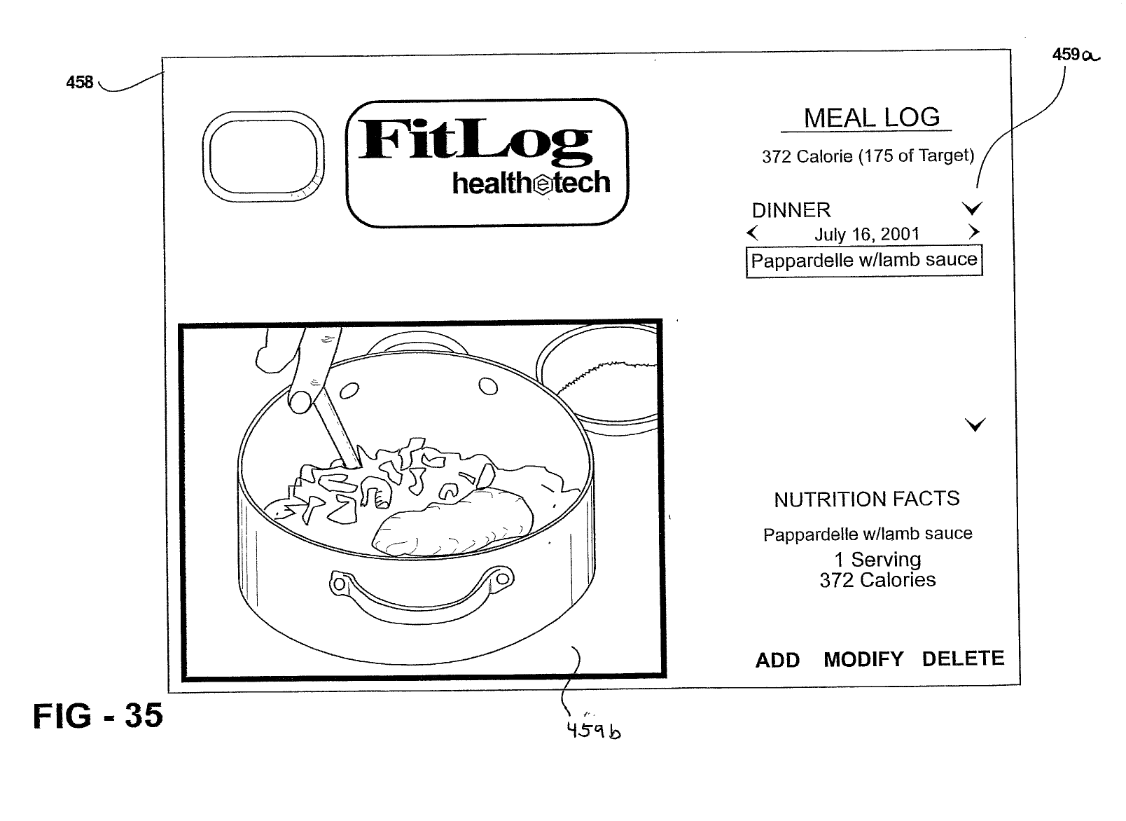

If the user 302 selects a meal log option, the methodology advances to block 425d and a meal log page is displayed on the television screen 306, as shown in FIG. 35 at 458. For example, the meal log option provides an opportunity for the user 302 to select meals for a predetermined time period, such as dinner, as shown at 459a. The user 302 can select a meal entree from a predetermined list of entrees maintained in the computer database, and determine whether to prepare and eat the meal, depending on whether the meal fits their goals for the day. For example, the user 302 can use the meal log to plan their daily meals to ensure that the meals they consume correspond with their personal fitness goals. The user 302 selects a particular mealtime, such as dinner, as well as a dinner entree, and is provided with information such as nutritional value of the selected meal, includes caloric, fat and vitamin analysis. The user 302 is provided with other options, such as to search for a food from a page listing a predetermined list of foods organized by food group. The user 302 can add the selected food to the meal log, modify a meal log entree or delete an existing meal log entree. Advantageously, the user can view a page listing the nutritional information associated with the food and simultaneously view a television program on how to prepare the food, as shown at 459b.

If the user selects the message option, the methodology advances to block 425e. In block 425e, the user 302 and personal fitness trainer 332 can interactively communicate with each other. For example, the personal fitness trainer 332 sends the user 302 a message with motivational advice. It is contemplated that the user 302 and personal fitness trainer 332 can interactively communicate using a known communication means, such as e-mail or instant messaging or the like.

It should be appreciated that the personal fitness program may also include other options, as described with respect to the balance log weight management program. For example, a menu option presents the user 302 with a page continuing a menu of healthy food choices selected by the personal fitness trainer 332 for the user 302.

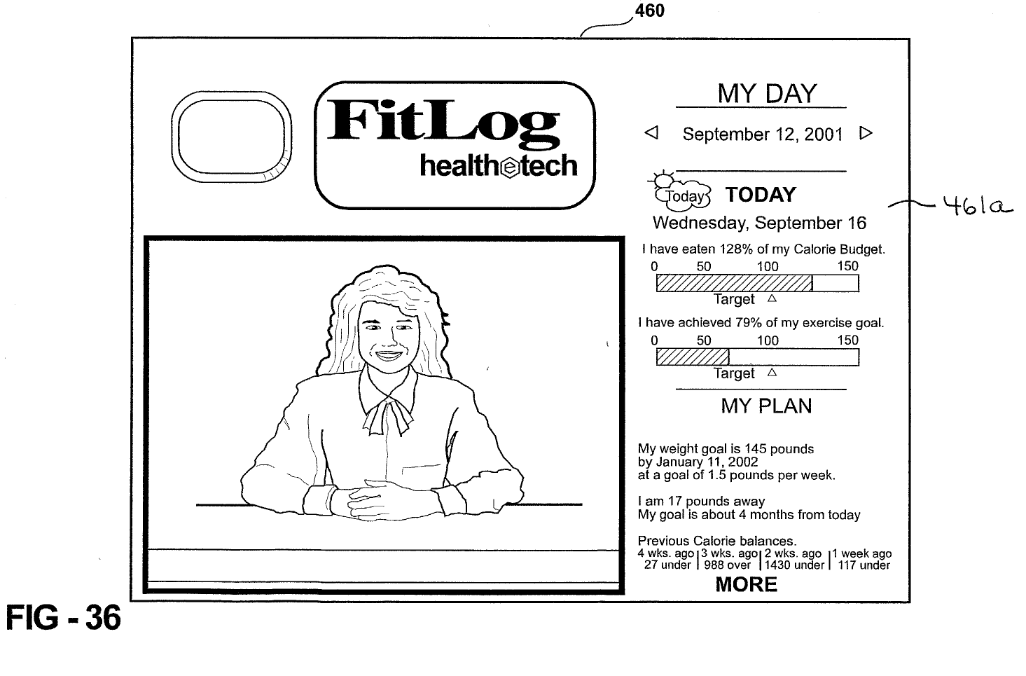

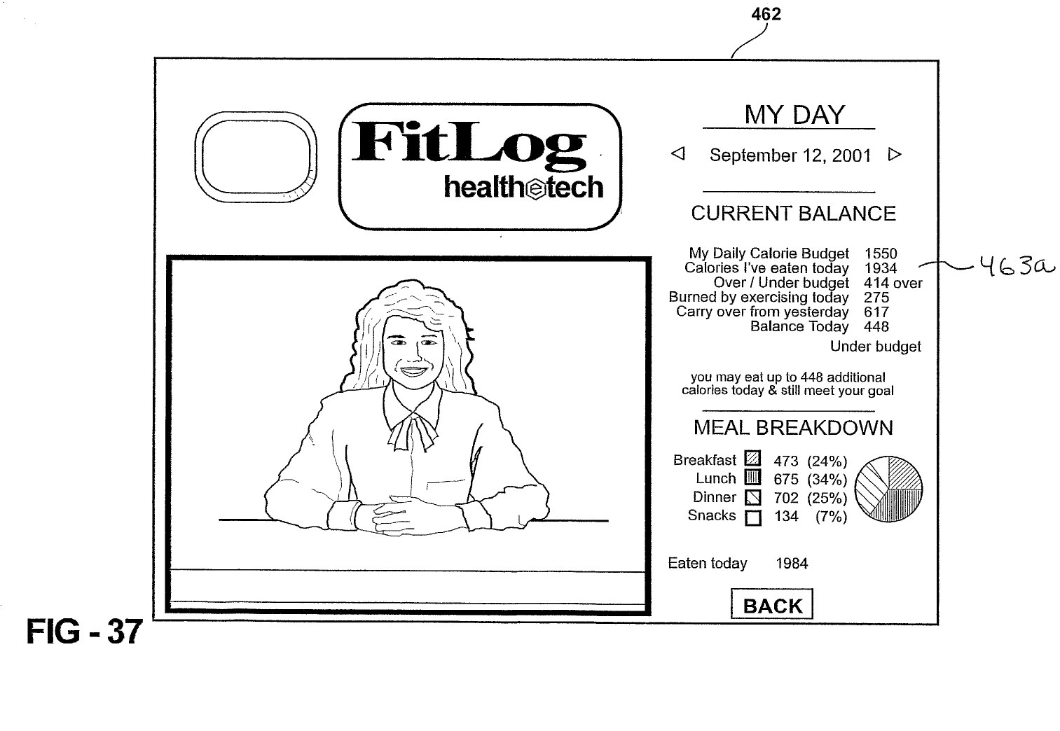

If the user 302 selects a summary option, the methodology advances to block 425f. In block 425f the user 302 is provided with a page containing a summary of the information related to their personal fitness training program as shown at 460 in FIG. 36. For example, the user's day is summarized in a personal fitness log, as shown at 461a. The fitness log is used by the personal fitness trainer 332 in evaluating the user's progress towards achieving their health management goals. The personal fitness trainer 332 modifies the program based on the user's progress. For example, the user 302 is provided with a summary of food consumed, percent of calorie budget for the day already eaten, percent of exercise goal reached, activity level, and caloric availability. In addition, the user 302 is presented with information regarding weight goals, and progress towards the weight goal for that day, or a previous day. The personal fitness training summary can also be presented on a page as shown in FIG. 37 at 462 with the summary in a graphical format as shown at 463a.

If the user 302 selects a my forecast option, the methodology advances to block 425g and user 302 is presented with a page containing a summary of information relating to how well the user is doing with respect to their personal training goals over a predetermined period of time.

If the user selects an account option, the methodology advances to block 425h and user 302 is presented with a page containing a summary of information relevant to their personal fitness training program. For example, the user 302 is presented with information such as personal details, including height, weight, age or other such information. The information may also include a summary of initial conditions, targets and goals, as previously described.

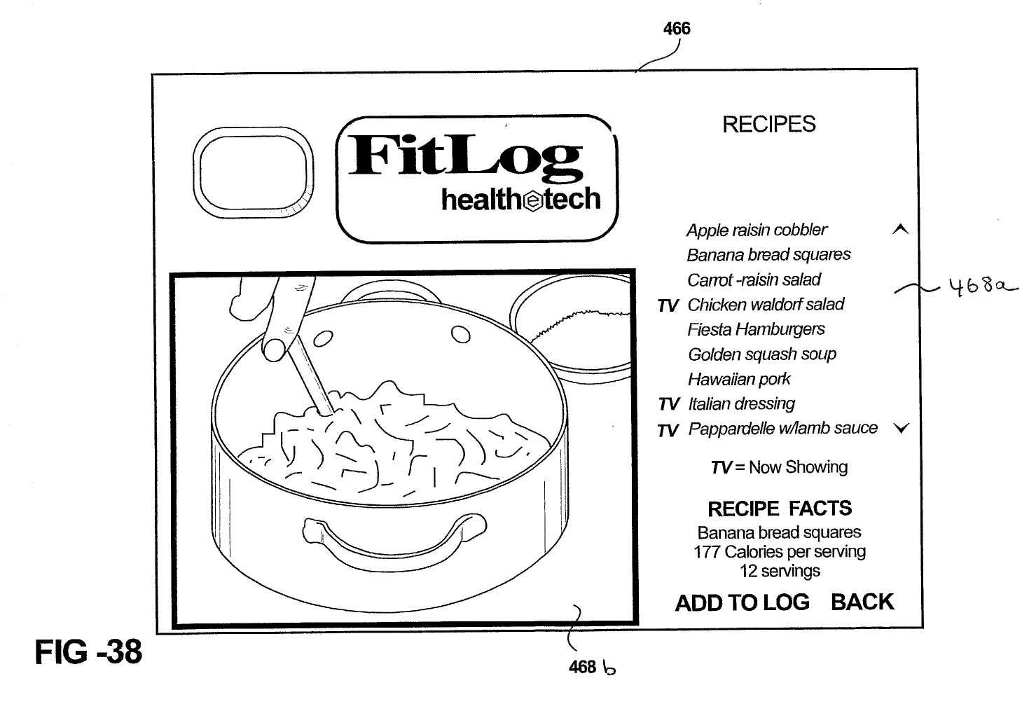

If the user 302 selects a recipe option, the methodology advances to block 425i and the user 302 is presented with a page as shown in FIG. 38 at 466 containing titles of recipes, as shown at 468a. Preferably, the recipes are maintained in a recipe database on the interactive television computer system 326. For example, the user 302 can search the recipe database for a particular recipe to view more specific information regarding how to prepare the recipe. The user 302 may also have the option of adding the recipe to the user's balance log weight management program, previously described. The user 302 may interactively select to view a recipe being demonstrated on a program simultaneously displayed on the screen 306 of the television set 304, as shown at 468b.

If the user 302 selects a preference option, the methodology advances to block 425j and user 302 is presented with a page on the interactive television 304 that allows them to view, establish or modify user preferences with respect to their personal fitness training program. Examples of user preferences include type of food or exercise or lifestyle or the like.

If the user 302 selects an exit option, the methodology advances to block 430 and the personal fitness training program log window is closed on the screen 306 of the television set 304. Advancing to block 435, the personal fitness trainer 332 periodically evaluates the diet and exercise information supplied by the user 302, and updates the user's personal fitness training program accordingly, to assist the user in achieving their health management goals. Preferably, the personal fitness training program is maintained in a database accessible to the user 302 via the interactive television communications network 322. The methodology advances to block 440 and the user 302 uses the personal fitness training program to plan and carry out their day, as part of their personal health management program. The methodology advances to block 445 and ends.

Returning to block 410, if the user 302 is not identified, the user 302 is provided an error message, and the methodology ends and the screen returns to television programming.

The present invention has been described in an illustrative manner. It is to be understood that the terminology, which has been used, is intended to be in the nature of words of description rather than of limitation.

Many modifications and variations of the present invention are possible in light of the above teachings. Therefore, within the scope of the appended claims, the present invention may be practiced other than as specifically described.

1. A system of personal fitness training using interactive television comprising:
an entertainment device having a display screen;
a control means in communication with said entertainment device and having a signal receiver and transmitter, and a microprocessor for controlling the entertainment device;
a remote control device having a user input mechanism, wherein a user input signal is transmitted to said control means via a communication link;
an interactive television network in communication with said control means via a communications network; and
a computer system in communication with said interactive television network, wherein said interactive television network transmits a signal via the communication network representing a personal fitness training program for the user, that is processed and displayed on the display screen of said entertainment device, the user interactively participates in the personal fitness training program using the remote control device, control means and entertainment device and the user uses the remote control device, control means and entertainment device to interact via the interactive television network.

2. A system as set forth in claim 1 wherein a personal fitness trainer develops the personal fitness training program for the user and the user uses the remote control device, control means and entertainment device to interact with the personal fitness trainer via the interactive television network.

3. A system as set forth in claim 1 wherein said entertainment device is a television set.

4. A system as set forth in claim 1 wherein said control means is a set top box.

5. A system as set forth in claim 1 wherein the personal fitness training program is a software program that provides the user with a window displayed on a portion of the display screen concurrent with a televised program, based on the user's transactional selections.

6. A system as set forth in claim 1 wherein the personal fitness training program is a software program that provides the user with a window displayed on a portion of the display screen and television programming relating to the window displayed on the other portion of the display screen, based on the user's transactional selections.

7. A system as set forth in claim 1 wherein said interactive television network includes a computer system having a memory and a processor for implementing the personal fitness training program.

8. A system as set forth in claim 7 wherein said interactive television network computer system is in communication with a personal fitness trainer computer system via the Internet, and the personal fitness training program is a software program maintained and implemented by the personal fitness trainer computer system.

9. A system as set forth in claim 1 wherein said personal fitness training program correlates the user's resting metabolism with caloric intake and activity level to determine the user's caloric expenditure and then determines the user's caloric balance from caloric intake and caloric expenditure.

10. A system of personal fitness training using interactive television comprising:
an entertainment device having a display screen;
a control means in communication with said entertainment device and having a signal receiver and transmitter, and a microprocessor for controlling the entertainment device;
a remote control device having a user input mechanism, wherein a user input signal is transmitted to said control means via a communication link;
an interactive television network in communication with said control means via a communications network; and
a personal fitness trainer computer system in communication with the interactive television network via a communications network wherein the personal fitness trainer uses the personal trainer computer system and interactive television network to transmit a signal representing a personal fitness training program developed by the personal fitness trainer for the user that correlates the user's resting metabolism with caloric intake and activity level to determine the user's caloric expenditure and then determines the user's caloric balance from caloric intake and caloric expenditure to the user, that is processed and displayed on the display screen of the entertainment device, the user interactively participates in the personal fitness training program using the remote control device, control means and entertainment device, and the user uses the remote control device, control means and entertainment device to interact with the personal fitness trainer via the interactive television network.

11. A method of personal fitness training using interactive television comprising:
selecting a personal fitness training program for a user, by the user using a remote control device in communication with a control means operatively in communication with an entertainment device, wherein the control means is operatively in communication with an interactive television network;
providing the user with a window on a display screen for the entertainment device containing a predetermined list of transactional selections for the personal fitness training program;
selecting a transaction by the user;
processing the user transactional selection by the interactive television network and providing a signal representing the personal fitness training program to the control means via the communications network that is processed and displayed on the display screen of the entertainment device; and
interactively using the personal fitness training program information displayed on the display screen to participate in the personal fitness training program via the interactive television network using the remote control device, control means and entertainment device.

12. A method as set forth in claim 11 including the step of identifying the user as a participant in the personal fitness training program by the interactive television network.

13. A method as set forth in claim 11 wherein said step of providing the user with transactional selections includes providing the user with a search option for searching an interactive television network computer database containing information relating to the personal fitness training program.

14. A method as set forth in claim 11 wherein said step of providing the user with transactional selections includes providing the user with a meal log option, wherein the user selects a meal entree from a predetermined list of meal entrees maintained in an interactive television network computer database to be included in the user's caloric intake.

15. A method as set forth in claim 11 wherein said step of providing the user with transactional selections includes providing the user with a meal log option, wherein the user modifies a predetermined list of meal entrees maintained in an interactive television network computer database for the personal fitness training program.

16. A method as set forth in claim 15 wherein said step of providing the user with transactional selections includes providing the user with a meal log option, and the user selects to view a television broadcast on the display screen relating to a meal entree from a predetermined list of meal entrees maintained in an interactive television network computer database for the personal fitness training program.

17. A method as set forth in claim 11 wherein said step of providing the user with transactional selections includes providing the user with an exercise log option for the personal fitness training program, wherein the user selects an exercise from a predetermined list of exercises maintained in an interactive television network computer database, for inclusion in determining the user's activity level.

18. A method as set forth in claim 17 wherein said step of providing the user with transactional selections includes providing the user with a exercise log option, and the user selects to view a television broadcast on the display screen relating to an exercise selected from a predetermined list of exercises maintained in an interactive television network computer database for the personal fitness training program.

19. A method as set forth in claim 11 wherein said step of providing the user with transactional selections includes providing the user with a summary option, wherein the user is provided with a summary of information on the display screen relating to the personal fitness training program from the interactive television network computer database.

20. A method as set forth in claim 19 wherein the summary includes caloric intake, caloric expenditure, and caloric availability for a predetermined period of time.

21. A method as set forth in claim 11 wherein said step of providing the user with transactional selections includes providing the user with a recipe option, wherein the user selects a recipe from a predetermined list of recipes maintained in an interactive television network computer database for the personal fitness training program and the user is provided with information regarding the recipe on the display screen.

22. A method as set forth in claim 21 wherein said step of providing the user with transactional selections includes providing the user with a recipe option, wherein the user selects to view a television broadcast on the display screen relating to the recipe from a predetermined list of recipes.

23. A method as set forth in claim 11 wherein the personal fitness training program is developed by a personal fitness trainer for the user.
